SEQUENCE LISTING (58 sequences)

SEQ ID NO: 1 - protein, 215 aa, Homo sapiens
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEEDEEDEEDEEEEEDEEDEDEEEDDDDE

SEQ ID NO: 2 - protein, 215 aa, Mus musculus
MGKGDPKKPRGKMSSYAFFVQTCREEHKKKHPDASVNFSEFSKKCSERWKTMSAKEKGKFEDMAKADKARYEREMKTYIPPKGETKKKFKDPNAPKRPPSAFFLFCSEYRPKIKGEHPGLSIGDVAKKLGEMWNNTAADDKQPYEKKAAKLKEKYEKDIAAYRAKGKPDAAKKGVVKAEKSKKKKEEEDDEEDEEDEEEEEEEEDEDEEEDDDDE

SEQ ID NO: 3 - protein, 209 aa, Homo sapiens
MGKGDPNKPRGKMSSYAFFVQTCREEHKKKHPDSSVNFAEFSKKCSERWKTMSAKEKSKFEDMAKSDKARYDREMKNYVPPKGDKKGKKKDPNAPKRPPSAFFLFCSEHRPKIKSEHPGLSIGDTAKKLGEMWSEQSAKDKQPYEQKAAKLKEKYEKDIAAYRAKGKSEAGKKGPGRPTGSKKKNEPEDEEEEEEEEDEDEEEEDEDEE

SEQ ID NO: 4 - protein, 54 aa, Homo sapiens
PDASVNFSEFSKKCSERWKTMSAKEKGKFEDMAKADKARYEREMKTYIPPKGET

SEQ ID NO: 5 - protein, 69 aa, Homo sapiens
NAPKRPPSAFFLFCSEYRPKIKGEHPGLSIGDVAKKLGEMWNNTAADDKQPYEKKAAKLKEKYEKDIAA

SEQ ID NO: 6 - DNA, 22 bp, Homo sapiens
gatgggcaaaggagatcctaag

SEQ ID NO: 7 - DNA, 29 bp, Homo sapiens
gcggccgcttattcatcatcatcatcttc

SEQ ID NO: 8 - DNA, 22 bp, Homo sapiens
gatgggcaaaggagatcctaag

SEQ ID NO: 9 - DNA, 32 bp, Homo sapiens
gcggccgctcacttgcttttttcagccttgac

SEQ ID NO: 10 - DNA, 21 bp, Homo sapiens
gagcataagaagaagcaccca

SEQ ID NO: 11 - DNA, 32 bp, Homo sapiens; identical to SEQ ID NO: 9

SEQ ID NO: 12 - DNA, 24 bp, Homo sapiens
aagttcaaggatcccaatgcaaag

SEQ ID NO: 13 - DNA, 32 bp, Homo sapiens
gcggccgctcaatatgcagctatatccttttc

SEQ ID NO: 14 - DNA, 22 bp, Homo sapiens
gatgggcaaaggagatcctaag

SEQ ID NO: 15 - DNA, 24 bp, Homo sapiens
tcacttttttgtctcccctttggg

SEQ ID NO: 16 - protein, 20 aa, Homo sapiens
NAPKRPPSAFFLFCSEYRPK

SEQ ID NO: 17 - protein, 54 aa, Homo sapiens
PDSSVNFAEFSKKCSERWKTMSAKEKSKFEDMAKSDKARYDREMKNYVPPKGDK

SEQ ID NO: 18 - protein, 216 aa, Homo sapiens [sequence omitted]

SEQ ID NO: 19 - protein, 182 aa, Homo sapiens
MGKGDPKKPTGKMSSYAFFVQTCREEHKKKHPDASVNFSEFSKKCSERWKTMSAKEKGKFEDMAKADKARYEREMKTYIPPKGETKKKFKDPNAPKRLPSAFFLFCSEYRPKIKGEHPGLSIGDVAKKLGEMWNNTAADDKQPYEKKAAKLKEKYEKDIAAYRAKGKPDAAKKGVVKAEKSK

SEQ ID NO: 20 - protein, 74 aa, Homo sapiens
FKDPNAPKRLPSAFFLFCSEYRPKIKGEHPGLSIGDVAKKLGEMWNNTAADDKQPYEKKAAKLKEKYEKDIAAY

SEQ ID NO: 21 - protein, 85 aa, Homo sapiens
MGKGDPKKPTGKMSSYAFFVQTCREEHKKKHPDASVNFSEFSKKCSERWKTMSAKEKGKFEDMAKADKARYEREMKTYIPPKGET

SEQ ID NO: 22 - protein, 77 aa, Homo sapiens
PTGKMSSYAFFVQTCREEHKKKHPDASVNFSEFSKKCSERWKTMSAKEKGKFEDMAKADKARYEREMKTYIPPKGET

SEQ ID NO: 23 - protein, 20 aa, Homo sapiens
FKDPNAPKRLPSAFFLFCSE

SEQ ID NO: 24 - protein, 216 aa, Homo sapiens [sequence omitted]

SEQ ID NO: 25 - protein, 211 aa, Homo sapiens
MGKGDPKKPRGKMSSYAFFVQTCREEHKKKHSDASVNFSEFSNKCSERWKTMSAKEKGKFEDMAKADKTHYERQMKTYIPPKGETKKKFKDPNAPKRPPSAFFLFCSEYHPKIKGEHPGLSIGDVAKKLGEMWNNTAADDKQPGEKKAAKLKEKYEKDIAAYQAKGKPEAAKKGVVKAEKSKKKKEEEEDEEDEEDEEEEDEEDEEDDDDE

SEQ ID NO: 26 - protein, 188 aa, Homo sapiens
MGKGDPKKPRGKMSSYAFFVQTCREECKKKHPDASVNFSEFSKKCSERWKAMSAKDKGKFEDMAKVDKDRYEREMKTYIPPKGETKKKFEDSNAPKRPPSAFLLFCSEYCPKIKGEHPGLPISDVAKKLVEMWNNTFADDKQLCEKKAAKLKEKYKKDTATYRAKGKPDAAKKGVVKAEKSKKKKEEE

SEQ ID NO: 27 - protein, 205 aa, Homo sapiens [sequence omitted]

SEQ ID NO: 28 - protein, 80 aa, Homo sapiens
MGKGDPKKPRGKMSSCAFFVQTCWEEHKKQYPDASINFSEFSQKCPETWKTTIAKEKGKFEDMPKADKAHYEREMKTYIP

SEQ ID NO: 29 - protein, 80 aa, Homo sapiens
KQRGKMPSYVFCVQTCPEERKKKHPDASVNFSEFSKKCLVRGKTMSAKEKGQFEAMARADKARYEREMKTYIPPKGETKK

SEQ ID NO: 30 - protein, 86 aa, Homo sapiens
MGKRDPKQPRGKMSSYAFFVQTAQEEHKKKQLDASVSFSEFSKNCSERWKTMSVKEKGKFEDMAKADKACYEREMKIYPYLKGRQK

SEQ ID NO: 31 - protein, 70 aa, Homo sapiens
MGKGDPKKPREKMPSYAFFVQTCREAHKNKHPDASVNSSEFSKKCSERWKTMPTKQKGKFEDMAKADRAH

SEQ ID NO: 32 - DNA, 648 bp, Homo sapiens
atgggcaaaggagatcctaagaagccgacaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttatgaaagagaaatgaaaacctatatccctcccaaaggggagacaaaaaagaagttcaaggatcccaatgcacccaagaggcttccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatatagctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagaagatgaagaagaagatgatgatgatgaa

SEQ ID NO: 33 - DNA, 633 bp, Homo sapiens
atgggcaaaggagatcctaagaagccgagaggcaaaatgtcatcatatgcattttttgtgcaaacttgtcgggaggagcataagaagaagcactcagatgcttcagtcaacttctcagagttttctaacaagtgctcagagaggtggaagaccatgtctgctaaagagaaaggaaaatttgaggatatggcaaaggcggacaagacccattatgaaagacaaatgaaaacctatatccctcccaaaggggagacaaaaaagaagttcaaggatcccaatgcacccaagaggcctccttcggccttcttcctgttctgctctgagtatcacccaaaaatcaaaggagaacatcctggcctgtccattggtgatgttgcgaagaaactgggagagatgtggaataacactgctgcagatgacaagcagcctggtgaaaagaaggctgcgaagctgaaggaaaaatacgaaaaggatattgctgcatatcaagctaaaggaaagcctgaggcagcaaaaaagggagttgtcaaagctgaaaaaagcaagaaaaagaaggaagaggaggaagatgaggaagatgaagaggatgaggaggaggaagatgaagaagatgaagaagatgatgatgatgaa

SEQ ID NO: 34 - DNA, 564 bp, Homo sapiens
atgggcaaaggagaccctaagaagccgagaggcaaaatgtcatcatatgcattttttgtgcaaacttgtcgggaggagtgtaagaagaagcacccagatgcttcagtcaacttctcagagttttctaagaagtgctcagagaggtggaaggccatgtctgctaaagataaaggaaaatttgaagatatggcaaaggtggacaaagaccgttatgaaagagaaatgaaaacctatatccctcctaaaggggagacaaaaaagaagttcgaggattccaatgcacccaagaggcctccttcggcctttttgctgttctgctctgagtattgcccaaaaatcaaaggagagcatcctggcctgcctattagcgatgttgcaaagaaactggtagagatgtggaataacacttttgcagatgacaagcagctttgtgaaaagaaggctgcaaagctgaaggaaaaatacaaaaaggatacagctacatatcgagctaaaggaaagcctgatgcagcaaaaaagggagttgtcaaggctgaaaaaagcaagaaaaagaaggaagaggag

SEQ ID NO: 35 - DNA, 615 bp, Homo sapiens
atggacaaagcagatcctaagaagctgagaggtgaaatgttatcatatgcattttttgtgcaaacttgtcaggaggagcataagaagaagaacccagatgcttcagtcaagttctcagagtttttaaagaagtgctcagagacatggaagaccatttttgctaaagagaaaggaaaatttgaagatatggcaaaggcggacaaggcccattatgaaagagaaatgaaaacctatatccctcctaaaggggagaaaaaaaagaagttcaaggatcccaatgcacccaagaggcctcctttggcctttttcctgttctgctctgagtatcgcccaaaaatcaaaggagaacatcctggcctgtccattgatgatgttgtgaagaaactggcagggatgtggaataacaccgctgcagctgacaagcagttttatgaaaagaaggctgcaaagctgaaggaaaaatacaaaaaggatattgctgcatatcgagctaaaggaaagcctaattcagcaaaaaagagagttgtcaaggctgaaaaaagcaagaaaaagaaggaagaggaagaagatgaagaggatgaacaagaggaggaaaatgaagaagatgatgataaa

SEQ ID NO: 36 - DNA, 240 bp, Homo sapiens
atgggcaaaggagatcctaagaagccgagaggcaaaatgtcatcatgtgcattttttgtgcaaacttgttgggaggagcataagaagcagtacccagatgcttcaatcaacttctcagagttttctcagaagtgcccagagacgtggaagaccacgattgctaaagagaaaggaaaatttgaagatatgccaaaggcagacaaggcccattatgaaagagaaatgaaaacctatataccc

SEQ ID NO: 37 - DNA, 240 bp, Homo sapiens
aaacagagaggcaaaatgccatcgtatgtattttgtgtgcaaacttgtccggaggagcgtaagaagaaacacccagatgcttcagtcaacttctcagagttttctaagaagtgcttagtgagggggaagaccatgtctgctaaagagaaaggacaatttgaagctatggcaagggcagacaaggcccgttacgaaagagaaatgaaaacatatatccctcctaaaggggagacaaaaaaa

SEQ ID NO: 38 - DNA, 258 bp, Homo sapiens
atgggcaaaagagaccctaagcagccaagaggcaaaatgtcatcatatgcattttttgtgcaaactgctcaggaggagcacaagaagaaacaactagatgcttcagtcagtttctcagagttttctaagaactgctcagagaggtggaagaccatgtctgttaaagagaaaggaaaatttgaagacatggcaaaggcagacaaggcctgttatgaaagagaaatgaaaatatatccctacttaaaggggagacaaaaa

SEQ ID NO: 39 - DNA, 211 bp, Homo sapiens
atgggcaaaggagaccctaagaagccaagagagaaaatgccatcatatgcattttttgtgcaaacttgtagggaggcacataagaacaaacatccagatgcttcagtcaactcctcagagttttctaagaagtgctcagagaggtggaagaccatgcctactaaacagaaaggaaaattcgaagatatggcaaaggcagacagggcccata

SEQ ID NO: 40 - protein, 54 aa, Homo sapiens; identical to SEQ ID NO: 4

SEQ ID NO: 41 - protein, 53 aa, Homo sapiens
DSSVNFAEFSKKCSERWKTMSAKEKSKFEDMAKSDKARYDREMKNYVPPKGDK

SEQ ID NO: 42 - protein, 54 aa, Homo sapiens
PEVPVNFAEFSKKCSERWKTVSGKEKSKFDEMAKADKVRYDREMKDYGPAKGGK

SEQ ID NO: 43 - protein, 54 aa, Homo sapiens; identical to SEQ ID NO: 4

SEQ ID NO: 44 - protein, 54 aa, Homo sapiens
SDASVNFSEFSNKCSERWKTMSAKEKGKFEDMAKADKTHYERQMKTYIPPKGET

SEQ ID NO: 45 - protein, 54 aa, Homo sapiens
PDASVNFSEFSKKCSERWKAMSAKDKGKFEDMAKVDKADYEREMKTYIPPKGET

SEQ ID NO: 46 - protein, 54 aa, Homo sapiens
PDASVKFSEFLKKCSETWKTIFAKEKGKFEDMAKADKAHYEREMKTYIPPKGEK

SEQ ID NO: 47 - protein, 54 aa, Homo sapiens
PDASINFSEFSQKCPETWKTTIAKEKGKFEDMAKADKAHYEREMKTYIPPKGET

SEQ ID NO: 48 - protein, 38 aa, Homo sapiens
PDASVNSSEFSKKCSERWKTMPTKQGKFEDMAKADRAH

SEQ ID NO: 49 - protein, 54 aa, Homo sapiens
PDASVNFSEFSKKCLVRGKTMSAKEKGQFEAMARADKARYEREMKTYIPPKGET

SEQ ID NO: 50 - protein, 54 aa, Homo sapiens
LDASVSFSEFSNKCSERWKTMSVKEKGKFEDMAKADKACYEREMKIYPYLKGRQ

SEQ ID NO: 51 - protein, 74 aa, Homo sapiens
FKDPNAPKRPPSAFFLFCSEYRPKIKGEHPGLSIGDVAKKLGEMWNNTAADDKQPYEKKAAKLKEKYEKDIAAY

SEQ ID NO: 52 - protein, 74 aa, Homo sapiens
KKDPNAPKRPPSAFFLFCSEHRPKIKSEHPGLSIGDTAKKLGEMWSEQSAKDKQPYEQKAAKLKEKYEKDIAAY

SEQ ID NO: 53 - protein, 74 aa, Homo sapiens; identical to SEQ ID NO: 20

SEQ ID NO: 54 - protein, 74 aa, Homo sapiens
FKDPNAPKRPPSAFFLFCSEYHPKIKGEHPGLSIGDVAKKLGEMWNNTAADDKQPGEKKAAKLKEKYEKDIAAY

SEQ ID NO: 55 - protein, 74 aa, Homo sapiens
FKDSNAPKRPPSAFLLFCSEYCPKIKGEHPGLPISDVAKKLVEMWNNTFADDKQLCEKKAAKLKEKYKKDTATY

SEQ ID NO: 56 - protein, 74 aa, Homo sapiens
FKDPNAPKRPPSAFFLFCSEYRPKIKGEHPGLSIGDVVKKLAGMWNNTAAADKQFYEKKAAKLKEKYKKDIAAY

SEQ ID NO: 57 - protein, 84 aa, Homo sapiens
GKGDPKKPRGKMSSYAFFVQTCREEHKKKHPDASVNFSEFSKKCSERWKTMSAKEKGKFEDMAKADKARYEREMKTYIPPKGET

SEQ ID NO: 58 - protein, 92 aa, Homo sapiens
FKDPNAPKRPPSAFFLFCSEYRPKIKGEHPGLSIGDVAKKLGEMWNNTAADDKQPYEKKAAKLKEKYEKDIAAYRAKGKPDAAKKGVVKAEK

(19) United States
(12) Patent Application Publication
(10) Pub. No.: US 2004/0156851 A1
(43) Pub. Date: Aug. 12, 2004

(54) HMGB1 combination therapies

(75) Inventor: Walter Newman, Boston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Critical Therapeutics, Inc., Cambridge, MA

(21) Appl. No.: 10/717,984

(22) Filed: Nov. 20, 2003

Related U.S. Application Data
(60) Provisional application No. 60/427,846, filed on Nov. 20, 2002.

(51) Int. Cl. A61K 39/395

(52) U.S. Cl. 424/145.1

(57) ABSTRACT

Compositions and methods are disclosed for treating a condition characterized by activation of an inflammatory cytokine cascade in a patient. The compositions comprise an HMGB A box and an inhibitor of TNF biological activity, or an antibody that binds an HMGB polypeptide or biologically active fragment thereof and an inhibitor of TNF biological activity. The methods comprise treating a cell or a patient with sufficient amounts of the composition to inhibit the release of the proinflammatory cytokine, or to inhibit the inflammatory cytokine cascade.

### RELATED APPLICATIONS

This application claims the benefit of U.S. Provisional Application No. 60/427,846, filed Nov. 20, 2002, the entire teachings of which are incorporated herein by reference.

### BACKGROUND OF THE INVENTION

Inflammation is often induced by proinflammatory cytokines, such as tumor necrosis factor (TNF), interleukin (IL)-1α, IL-1β, IL-6, platelet-activating factor (PAF), macrophage migration inhibitory factor (MIF), and other compounds. These proinflammatory cytokines are produced by several different cell types, most importantly immune cells (for example, monocytes, macrophages and neutrophils), but also non-immune cells such as fibroblasts, osteoblasts, smooth muscle cells, epithelial cells, and neurons. These proinflammatory cytokines contribute to various disorders during the early stages of an inflammatory cytokine cascade.

The early proinflammatory cytokines (e.g., TNF, IL-1, etc.) mediate inflammation, and induce the late release of high mobility group box 1 (HMGB1; also known as HMG-1 and HMG1), a protein that accumulates in serum and mediates delayed lethality and further induction of early proinflammatory cytokines.

HMGB1 was first identified as the founding member of a family of DNA-binding proteins termed high mobility group box (HMGB) that are critical for DNA structure and stability. It was identified nearly 40 years ago as a ubiquitously expressed nuclear protein that binds double-stranded DNA without sequence specificity. The HMGB1 molecule has three domains: two DNA binding motifs termed HMGB A and HMGB B boxes, and an acidic carboxyl terminus. The two HMGB boxes are highly conserved 80 amino acid, L-shaped domains. HMGB boxes are also expressed in other transcription factors including the RNA polymerase I transcription factor human upstream-binding factor and lymphoid-specific factor.

Recent evidence has implicated HMGB1 as a cytokine mediator of a number of inflammatory conditions. The delayed kinetics of HMGB1 appearance during endotoxemia makes it a potentially good therapeutic target, but little is known about the molecular basis of HMGB1 signaling and toxicity.

### SUMMARY OF THE INVENTION

The present invention is based on the discoveries that combination therapies involving agents that inhibit HMGB biological activity and agents that inhibit TNF biological activity can be used for the treatment of conditions characterized by activation of the inflammatory cytokine cascade. Agents that inhibit HMGB biological activity include the HMGB A box, which serves as a competitive inhibitor of HMGB proinflammatory action, and antibodies to HMGB, for example, the HMGB B box, which has the predominant proinflammatory activity of HMGB.

Accordingly, the present invention is directed to a pharmaceutical composition comprising a polypeptide comprising a high mobility group box (HMGB) A box or a fragment or variant thereof that can inhibit release of a proinflammatory cytokine from a cell treated with a high mobility group box (HMGB) protein and an agent that inhibits TNF biological activity, where the agent is selected from the group consisting of infliximab, etanercept, adalimumab, CDP870, CDP571, Lenercept, and Thalidomide, in a pharmaceutically acceptable carrier. The HMGB A box is preferably a vertebrate HMGB A box, for example, a mammalian HMGB A box, more preferably, a mammalian HMGB1 A box, for example, a human HMGB1 A box, and most preferably, the HMGB1 A box comprising or consisting of the sequence of SEQ ID NO:4, SEQ ID NO:22, or SEQ ID NO:57.

In another embodiment, the invention is a pharmaceutical composition comprising an antibody that binds an HMGB polypeptide or a biologically active fragment thereof, for example, an HMGB B box polypeptide or biologically active fragment thereof, and an agent that inhibits TNF biological activity, where the agent is selected from the group consisting of infliximab, etanercept, adalimumab, CDP870, CDP571, Lenercept, and Thalidomide, in a pharmaceutically acceptable carrier.

In another embodiment, the invention is a method of treating a condition in a patient characterized by activation of an inflammatory cytokine cascade comprising administering to the patient a composition comprising a polypeptide comprising a high mobility group box (HMGB) A box or a fragment or variant thereof that can inhibit release of a proinflammatory cytokine from a cell treated with high mobility group box (HMGB) protein and an agent that inhibits TNF biological activity, where the agent is selected from the group consisting of infliximab, etanercept, adalimumab, CDP870, CDP571, Lenercept, and Thalidomide.

In still another embodiment, the invention is a method of treating a condition in a patient characterized by activation of an inflammatory cytokine cascade comprising administering to the patient a composition comprising an antibody that binds an HMGB polypeptide or a biologically active fragment thereof, for example, an HMGB B box polypeptide or a biologically active fragment thereof, and an agent that inhibits TNF biological activity, where the agent is selected from the group consisting of infliximab, etanercept, adalimumab, CDP870, CDP571, Lenercept, and Thalidomide.

### BRIEF DESCRIPTION OF THE DRAWINGS

FIG. 1 is a schematic representation of HMGB1 mutants and their activity in TNF release (pg/ml).

FIG. 2A is a histogram showing the effect of 0 μg/ml, 0.01 μg/ml, 0.1 μg/ml, 1 μg/ml or 10 μg/ml of HMGB B box on TNF release (pg/ml) in RAW 264.7 cells.

FIG. 2B is a histogram showing the effect of 0 μg/ml, 0.01 μg/ml, 0.1 μg/ml, 1 μg/ml or 10 μg/ml of HMGB B box on IL-1β release (pg/ml) in RAW 264.7 cells.

FIG. 2C is a histogram showing the effect of 0 μg/ml, 0.01 μg/ml, 0.1 μg/ml, 1 μg/ml or 10 μg/ml of HMGB B box on IL-6 release (pg/ml) in RAW 264.7 cells.

FIG. 2D a scanned image of a blot of an RNAse protection assay, showing the effect of HMGB B box (at 0 hours, 4 hours, 8 hours, or 24 hours after administration) or vector alone (at 4 hours after administration) on TNF mRNA expression in RAW 264.7 cells.

FIG. 2E is a histogram of the effect of HMGB1 B box on TNF protein release (pg/ml) from RAW 264.7 cells at 0 hours, 4 hours, 8 hours, 24 hours, 32 hours or 48 hours after administration.

FIG. 2F is a histogram of the effect of vector on TNF protein release (pg/ml) from RAW 264.7 cells at 0 hours, 4 hours, 8 hours, 24 hours, 32 hours or 48 hours after administration.

FIG. 3 is a schematic representation of HMGB1 B box mutants and their activity in TNF release (pg/ml).

FIG. 4A is a graph of the effect of 0 μg/ml, 5 μg/ml, 10 μg/ml, or 25 μg/ml of HMGB1 A box protein on the release of TNF (as a percent of HMG1 mediated TNF release alone) from RAW 264.7 cells.

FIG. 4B is a histogram of the effect of HMGB1 (0 or 1.5 μg/ml), HMGB1 A box (0 or 10 μg/ml), or vector (0 or 10 μg/ml), alone, or in combination, on the release of TNF (as a percent of HMG-1 mediated TNF release alone) from RAW 264.7 cells.

FIG. 5A is a graph of binding of ¹²⁵I-HMGB1 binding to RAW 264.7 cells (CPM/well) over time (minutes).

FIG. 5B is a histogram of the binding of ¹²⁵I-HMGB1 in the absence of unlabeled HMGB1 or HMG1 A box for 2 hours at 4° C. (Total), or in the presence of 5,000 molar excess of unlabeled HMGB1 (HMGB1) or A box (A box), measured as a percent of the total CPM/well.

FIG. 6 is a histogram of the effects of HMGB1 (HMG-1; 0 μg/ml or 1 μg/ml) or HMGB1 B box (B Box; 0 μg/ml or 10 μg/ml), alone or in combination with anti-B box antibody (25 μg/ml or 100 μg/ml) or IgG (25 μg/ml or 100 μg/ml) on TNF release from RAW 264.7 cells (expressed as a percent of HMG1 mediated TNF release alone).

FIG. 7A is a scanned image of a hematoxylin and eosin stained kidney section obtained from an untreated mouse.

FIG. 7B is a scanned image of a hematoxylin and eosin stained kidney section obtained from a mouse administered HMGB1 B box.

FIG. 7C is a scanned image of a hematoxylin and eosin stained myocardium section obtained from an untreated mouse.

FIG. 7D is a scanned image of a hematoxylin and eosin stained myocardium section obtained from a mouse administered HMGB1 B box.

FIG. 7E is a scanned image of a hematoxylin and eosin stained lung section obtained from an untreated mouse.

FIG. 7F is a scanned image of a hematoxylin and eosin stained lung section obtained from a mouse administered HMGB1 B box.

FIG. 7G is a scanned image of a hematoxylin and eosin stained liver section obtained from an untreated mouse.

FIG. 7H is a scanned image of a hematoxylin and eosin stained liver section obtained from a mouse administered HMGB1 B box.

FIG. 7I is a scanned image of a hematoxylin and eosin stained liver section (high magnification) obtained from an untreated mouse.

FIG. 7J is a scanned image of a hematoxylin and eosin stained liver section (high magnification) obtained from a mouse administered HMGB1 B box.

FIG. 8 is a graph of the level of HMGB1 (ng/ml) in mice subjected to cecal ligation and puncture (CLP) over time (hours).

FIG. 9 is a graph of the effect of HMGB A Box (60 μg/mouse or 600 μg/mouse) or no treatment on survival of mice over time (days) after cecal ligation and puncture (CLP).

FIG. 10A is a graph of the effect of anti-HMGB1 antibody (dark circles) or no treatment (open circles) on survival of mice over time (days) after cecal ligation and puncture (CLP).

FIG. 10B is a graph of the effect of anti-HMGB1 B box antiserum (▪) or no treatment (*) on the survival (days) of mice administered lipopolysaccharide (LPS).

FIG. 11A is a histogram of the effect of anti-RAGE antibody or non-immune IgG on TNF release from RAW 264.7 cells treated with HMGB1 (HMG-1), lipopolysaccharide (LPS), or HMG1 B box (B box).

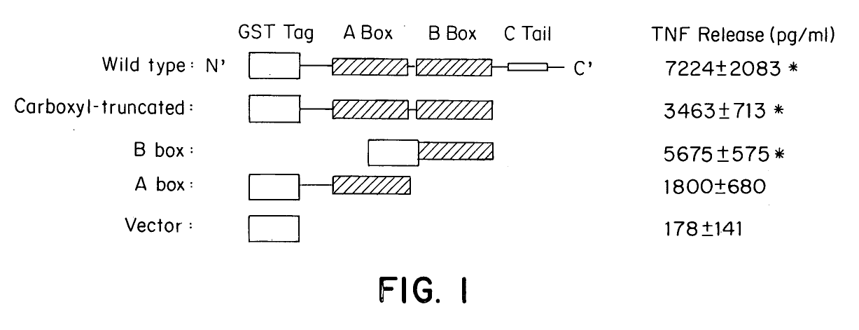

FIG. 1I B is a histogram of the effect of HMGB1 (HMG-1) or HMGB1 B box (B Box) polypeptide stimulation on activation of the NF-κB-dependent ELAM promoter (measured by luciferase activity) in RAW 264.7 cells co-transfected with a murine MyD 88-dominant negative (+MyD 88 DN) mutant (corresponding to amino acids 146-296), or empty vector (−MyD 88 DN). Data are expressed as the ratio (fold-activation) of average luciferase values from unstimulated and stimulated cells (subtracted for background)+SD.

FIG. 12A is the amino acid sequence of a human HMG1 polypeptide (SEQ ID NO:1).

FIG. 12B is the amino acid sequence of rat and mouse HMG1 (SEQ ID NO:2).

FIG. 12C is the amino acid sequence of human HMG2 (SEQ ID NO:3).

FIG. 12D is the amino acid sequence of a human, mouse, and rat HMG1 A box polypeptide (SEQ ID NO:4).

FIG. 12E is the amino acid sequence of a human, mouse, and rat HMG1 B box polypeptide (SEQ ID NO:5).

FIG. 12F is the nucleic acid sequence of a forward primer for human HMG1 (SEQ ID NO:6).

FIG. 12G is the nucleic acid sequence of a reverse primer for human HMG1 (SEQ ID NO:7).

FIG. 12H is the nucleic acid sequence of a forward primer for the carboxy terminus mutant of human HMG1 (SEQ ID NO:8).

FIG. 12I is the nucleic acid sequence of a reverse primer for the carboxy terminus mutant of human HMG1 (SEQ ID NO:9).

FIG. 12J is the nucleic acid sequence of a forward primer for the amino terminus plus B box mutant of human HMG1 (SEQ ID NO:10).

FIG. 12K is the nucleic acid sequence of a reverse primer for the amino terminus plus B box mutant of human HMG1 (SEQ ID NO:11).

FIG. 12L is the nucleic acid sequence of a forward primer for a B box mutant of human HMG1 (SEQ ID NO:12).

FIG. 12M is the nucleic acid sequence of a reverse primer for a B box mutant of human HMG1 (SEQ ID NO:13).

FIG. 12N is the nucleic acid sequence of a forward primer for the amino terminus plus A box mutant of human HMG1 (SEQ ID NO:14).

FIG. 12O is the nucleic acid sequence of a reverse primer for the amino terminus plus A box mutant of human HMG1 (SEQ ID NO:15).

FIG. 13 is a sequence alignment of HMGB1 polypeptide sequences from rat (SEQ ID NO:2), mouse (SEQ ID NO:2), and human (SEQ ID NO:18).

FIG. 14A is the nucleic acid sequence of HMG1L5 (formerly HMG1L10) (SEQ ID NO: 32) encoding an HMGB polypeptide.

FIG. 14B is the polypeptide sequence of HMG1L5 (formerly HMG1L10) (SEQ ID NO: 24) encoding an HMGB polypeptide.

FIG. 14C is the nucleic acid sequence of HMG1L1 (SEQ ID NO: 33) encoding an HMGB polypeptide.

FIG. 14D is the polypeptide sequence of HMG1L1 (SEQ ID NO: 25) encoding an HMGB polypeptide.

FIG. 14E is the nucleic acid sequence of HMG1L4 (SEQ ID NO: 34) encoding an HMGB polypeptide.

FIG. 14F is the polypeptide sequence of HMG1L4 (SEQ ID NO: 26) encoding an HMGB polypeptide.

FIG. 14G is the nucleic acid sequence of the HMG polypeptide sequence of the BAC clone RP11-395A23 (SEQ ID NO: 35).

FIG. 14H is the polypeptide sequence of the HMG polypeptide sequence of the BAC clone RP11-395A23 (SEQ ID NO: 27) encoding an HMGB polypeptide.

FIG. 14I is the nucleic acid sequence of HMG1L9 (SEQ ID NO: 36) encoding an HMGB polypeptide.

FIG. 14J is the polypeptide sequence of HMG1L9 (SEQ ID NO: 28) encoding an HMGB polypeptide.

FIG. 14K is the nucleic acid sequence of LOC122441 (SEQ ID NO: 37) encoding an HMGB polypeptide.

FIG. 14L is the polypeptide sequence of LOC122441 (SEQ ID NO: 29) encoding an HMGB polypeptide.

FIG. 14M is the nucleic acid sequence of LOC139603 (SEQ ID NO: 38) encoding an HMGB polypeptide.

FIG. 14N is the polypeptide sequence of LOC139603 (SEQ ID NO: 30) encoding an HMGB polypeptide.

FIG. 14O is the nucleic acid sequence of HMG1L8 (SEQ ID NO: 39) encoding an HMGB polypeptide.

FIG. 14P is the polypeptide sequence of HMG1L8 (SEQ ID NO: 31) encoding an HMGB polypeptide.

### DETAILED DESCRIPTION OF THE INVENTION

The practice of the present invention will employ, unless otherwise indicated, conventional techniques of cell culture, molecular biology, microbiology, cell biology, and immunology, which are well within the skill of the art. Such techniques are fully explained in the literature. See, e.g., Sambrook et al., 1989, “Molecular Cloning: A Laboratory Manual”, Cold Spring Harbor Laboratory Press; Ausubel et al. (1995), “Short Protocols in Molecular Biology”, John Wiley and Sons; Methods in Enzymology (several volumes); Methods in Cell Biology (several volumes), and Methods in Molecular Biology (several volumes).

The present invention is based on the discovery that inhibitors of TNF biological activity can be combined with HMGB A boxes and/or antibodies to HMGB1 to form pharmaceutical compositions for use in treating conditions characterized by activation of an inflammatory cytokine cascade in patients. The proinflammatory active domain of HMGB1 is the B box (and in particular, the first 20 amino acids of the B box), and antibodies specific to the B box inhibit proinflammatory cytokine release and inflammatory cytokine cascades, with results that can alleviate deleterious symptoms caused by inflammatory cytokine cascades (U.S. patent application Ser. No. 10/147,447, the entire teachings of which are incorporated by reference herein). In addition, the A box is a weak agonist of inflammatory cytokine release, and competitively inhibits the proinflammatory activity of the B box and of HMGB1 (U.S. patent application Ser. No. 10/147,447).

As used herein, an “HMGB polypeptide” or an “HMGB protein” is a substantially pure, or substantially pure and isolated polypeptide, that has been separated from components that naturally accompany it, or a synthetically or recombinantly produced polypeptide having the same amino acid sequence, and increases inflammation, and/or increases release of a proinflammatory cytokine from a cell, and/or increases the activity of the inflammatory cytokine cascade. In one embodiment, the HMGB polypeptide has one of the above biological activities. In another embodiment, the HMGB polypeptide has two of the above biological activities. In a third embodiment, the HMGB polypeptide has all three of the above biological activities.

Preferably, the HMGB polypeptide is a mammalian HMGB polypeptide, for example, a human HMGB1 polypeptide. Examples of an HMGB polypeptide include a polypeptide comprising or consisting of the sequence of SEQ ID NO:1, SEQ ID NO:2, SEQ ID NO:3, or SEQ ID NO:18. Preferably, the HMGB polypeptide contains a B box DNA binding domain and/or an A box DNA binding domain, and/or an acidic carboxyl terminus as described herein. Other examples of HMGB polypeptides are described in GenBank Accession Numbers AAA64970, AAB08987, P07155, AAA20508, S29857, P09429, NP_{—}002119, CAA31110, S02826, U00431, X67668, NP_{—}005333, NM_{—}016957, and J04179, the entire teachings of which are incorporated herein by reference. Additional examples of HMGB polypeptides include, but are not limited to mammalian HMG1 ((HMGB1) as described, for example, in GenBank Accession Number U51677), HMG2 ((HMGB2) as described, for example, in GenBank Accession Number M83665), HMG-2A ((HMGB3, HMG-4) as described, for example, in GenBank Accession Numbers NM_{—}005342 and NP_{—}005333), HMG14 (as described, for example, in GenBank Accession Number PO₅₁₁₄), HMG17 (as described, for example, in GenBank Accession Number X13546), HMG1 (as described, for example, in GenBank Accession Number L17131), and HMGY (as described, for example, in GenBank Accession Number M23618); nonmammalian HMG T1 (as described, for example, in GenBank Accession Number X02666) and HMG T2 (as described, for example, in GenBank Accession Number L32859) (rainbow trout); HMG-X (as described, for example, in GenBank Accession Number D30765) (Xenopus); HMG D (as described, for example, in GenBank Accession Number X71138) and HMG Z (as described, for example, in GenBank Accession Number X71139) (Drosophila); NHP10 protein (HMG protein homolog NHP 1) (as described, for example, in GenBank Accession Number Z48008) (yeast); non-histone chromosomal protein (as described, for example, in GenBank Accession Number 000479) (yeast); HMG ½ like protein (as described, for example, in GenBank Accession Number Z11540) (wheat, maize, soybean); upstream binding factor (UBF-1) (as described, for example, in GenBank Accession Number X53390); PMS1 protein homolog 1 (as described, for example, in GenBank Accession Number U13695); single-strand recognition protein (SSRP, structure-specific recognition protein) (as described, for example, in GenBank Accession Number M86737); the HMG homolog TDP-1 (as described, for example, in GenBank Accession Number M74017); mammalian sex-determining region Y protein (SRY, testis-determining factor) (as described, for example, in GenBank Accession Number X53772); fungal proteins: mat-1 (as described, for example, in GenBank Accession Number AB009451), ste 11 (as described, for example, in GenBank Accession Number X53431) and Mc 1; SOX 14 (as described, for example, in GenBank Accession Number AF107043), as well as SOX 1 (as described, for example, in GenBank Accession Number Y13436), SOX 2 (as described, for example, in GenBank Accession Number Z31560), SOX 3 (as described, for example, in GenBank Accession Number X71135), SOX 6 (as described, for example, in GenBank Accession Number AF309034), SOX 8 (as described, for example, in GenBank Accession Number AF226675), SOX 10 (as described, for example, in GenBank Accession Number AJ001183), SOX 12 (as described, for example, in GenBank Accession Number X73039) and SOX 21 (as described, for example, in GenBank Accession Number AF107044)); lymphoid specific factor (LEF-1)(as described, for example, in GenBank Accession Number X58636); T-cell specific transcription factor (TCF-1)(as described, for example, in GenBank Accession Number X59869); MTT1 (as described, for example, in GenBank Accession Number M62810); and SP100-HMG nuclear autoantigen (as described, for example, in GenBank Accession Number U36501).

Other examples of HMGB proteins are polypeptides encoded by HMGB nucleic acid sequences having GenBank Accession Numbers NG_{—}000897 (HMG1L5 (formerly HMG1L10)) (and in particular by nucleotides 150-797 of NG_{—}000897, as shown in FIGS. 14A and 14B); AF076674 (HMG1L1) (and in particular by nucleotides 1-633 of AF076674, as shown in FIGS. 14C and 14D; AF076676 (HMG1L4) (and in particular by nucleotides 1-564 of AF076676, as shown in FIGS. 14E and 14F); AC010149 (HMG sequence from BAC clone RP11-395A23) (and in particular by nucleotides 75503-76117 of AC010149), as shown in FIGS. 14G and 14H); AF165168 (HMG1L9) (and in particular by nucleotides 729-968 of AF165168, as shown in FIGS. 14I and 14J); XM_{—}063129 (LOC122441) (and in particular by nucleotides 319-558 of XM_{—}063129, as shown in FIGS. 14K and 14L); XM_{—}066789 (LOC139603) (and in particular by nucleotides 1-258 of XM_{—}066789, as shown in FIGS. 14M and 14N); and AF165167 (HMG1L8) (and in particular by nucleotides 456-666 of AF165167, as shown in FIGS. 140 and 14P).

The HMGB polypeptides of the present invention also encompass sequence variants. Variants include a substantially homologous polypeptide encoded by the same genetic locus in an organism, i.e., an allelic variant, as well as other variants. Variants also encompass polypeptides derived from other genetic loci in an organism, but having substantial homology to a polypeptide encoded by an HMGB nucleic acid molecule, and complements and portions thereof, or having substantial homology to a polypeptide encoded by a nucleic acid molecule comprising the nucleotide sequence of an HMGB nucleic acid molecule. Examples of HMGB nucleic acid molecules are known in the art and can be derived from HMGB polypeptides as described herein. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods. Preferably, the HMGB polypeptide has at least 60%, more preferably, at least 70%, 75%, 80%, 85%, or 90%, and most preferably at least 95% sequence identity to a sequence selected from SEQ ID NO:1, SEQ ID NO:2, SEQ ID NO:3, or SEQ ID NO:18, as determined using the BLAST program and parameters described herein and one of more of the biological activities of an HMGB polypeptide.

In other embodiments, the present invention is directed to an HMGB polypeptide fragment that has HMGB biological activity. By an “HMGB polypeptide fragment that has HMGB biological activity” or a “biologically active HMGB fragment” is meant a fragment of an HMGB polypeptide that has the activity of an HMGB polypeptide. An example of such an HMGB polypeptide fragment is the HMGB B box, as described herein. Biologically active HMGB fragments can be generated using standard molecular biology techniques and assaying the function of the fragment by determining if the fragment, when administered to a cell, increases release of a proinflammatory cytokine from the cell, compared to a suitable control, for example, using methods described herein.

As used herein, an “HMGB A box”, also referred to herein as an “A box”, is a substantially pure, or substantially pure and isolated polypeptide, that has been separated from components that naturally accompany it, and consists of an amino acid sequence that is less than a full length HMGB polypeptide and which has one or more of the following biological activities: inhibiting inflammation, and/or inhibiting release of a proinflammatory cytokine from a cell, and/or decreasing the activity of the inflammatory cytokine cascade. In one embodiment, the HMGB A box polypeptide has one of the above biological activities. In another embodiment, the HMGB A box polypeptide has two of the above biological activities. In a third embodiment, the HMGB A box polypeptide has all three of the above biological activities. Preferably, the HMGB A box has no more than 10%, 20%, 25%, 30%, 40%, 50%, 60%, 70%, 80%, or 90% of the biological activity of a full length HMGB polypeptide.

An HMGB A box is also an artificially or recombinantly produced polypeptide having the same amino acid sequence as the A box sequences described above. Preferably, the HMGB A box is a mammalian HMGB A box, for example, a human HMGB1 A box. The HMGB A box polypeptides of the present invention preferably comprise or consist of the sequence of SEQ ID NO:4, SEQ ID NO:22, or SEQ ID NO: 57, or the amino acid sequence in the corresponding region of an HMGB protein in a mammal. An HMGB A box often has no more than about 85 amino acids and no fewer than about 4 amino acids. Examples of polypeptides having A box sequences within them include, but are not limited to HMGB polypeptides described herein. The A box sequences in such polypeptides can be determined and isolated using methods described herein, for example, by sequence comparisons to A boxes described herein and testing for A box biological activity using methods described herein or other methods known in the art.

Examples of HMGB A box polypeptide sequences include the following sequences: PDASVNFSEF SKKCSERWKT MSAKEKGKFE DMAKADKARY EREMKTYIPP KGET (human HMGB1; SEQ ID NO: 40); DSSVNFAEF SKKCSERWKT MSAKEKSKFE DMAKSDKARY DREMKNYVPP KGDK (human HMGB2; SEQ ID NO: 41); PEVPVNFAEF SKKCSERWKT VSGKEKSKFD EMAKADKVRY DREMKDYGPA KGGK (human HMGB3; SEQ ID NO: 42); PDASVNFSEF SKKCSERWKT MSAKEKGKFE DMAKADKARY EREMKTYIPP KGET (HMG1L5 (formerly HMG1L10); SEQ ID NO: 43); SDASVNFSEF SNKCSERWKT MSAKEKGKFE DMAKADKTHY ERQMKTYIPP KGET (HMG1L1; SEQ ID NO: 44); PDASVNFSEF SKKCSERWKA MSAKDKGKFE DMAKVDKADY EREMKTYIPP KGET (HMG1L4; SEQ ID NO: 45); PDASVKFSEF LKKCSETWKT IFAKEKGKFE DMAKADKAHY EREMKTYIPP KGEK (HMG sequence from BAC clone RP1′-395A23; SEQ ID NO: 46); PDASINFSEF SQKCPETWKT TIAKEKGKFE DMAKADKAHY EREMKTYIPP KGET (HMG1L9; SEQ ID NO: 47); PDASVNSSEF SKKCSERWKTMPTKQGKFE DMAKADRAH (HMG1L8; SEQ ID NO: 48); PDASVNFSEF SKKCLVRGKT MSAKEKGQFE AMARADKARY EREMKTYIP PKGET (LOC122441; SEQ ID NO: 49); LDASVSFSEF SNKCSERWKT MSVKEKGKFE DMAKADKACY EREMKIYPYL KGRQ (LOC139603; SEQ ID NO: 50); and GKGDPKKPRG KMSSYAFFVQ TCREEHKKKH PDASVNFSEF SKKCSERWKT MSAKEKGKFE DMAKADKARY EREMKTYIPP KGET (human HMGB1 A box; SEQ ID NO: 57).

The HMGB A box polypeptides of the present invention also encompass sequence variants. Variants include a substantially homologous polypeptide encoded by the same genetic locus in an organism, i.e., an allelic variant, as well as other variants. Variants also encompass polypeptides derived from other genetic loci in an organism, but having substantial homology to a polypeptide encoded by an HMGB A box nucleic acid molecule, and complements and portions thereof, or having substantial homology to a polypeptide encoded by a nucleic acid molecule comprising the nucleotide sequence of an HMGB A box nucleic acid molecule. Examples of HMGB A box nucleic acid molecules are known in the art and can be derived from HMGB A polypeptides as described herein. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods. Preferably, an HMGB A box has at least 60%, more preferably, at least 70%, 75%, 80%, 85%, or 90%, and most preferably at least 95% sequence identity to an HMGB A box polypeptide described herein, for example, the sequence of SEQ ID NO:4, SEQ ID NO:22, or SEQ ID NO:57, as determined using the BLAST program and parameters described herein and one of more of the biological activities of an HMGB A box.

The present invention also features A box biologically active fragments. By an “A box fragment that has A box biological activity” or an “A box biologically active fragment” is meant a fragment of an HMGB A box that has the activity of an HMGB A box, as described herein. For example, the A box fragment can decrease release of a pro-inflammatory cytokine from a vertebrate cell, decrease inflammation, and/or decrease activity of the inflammatory cytokine cascade. A box fragments can be generated using standard molecular biology techniques and assaying the function of the fragment by determining if the fragment, when administered to a cell inhibits release of a proinflammatory cytokine from the cell, for example, using methods described herein. A box biologically active fragments can be used in the methods described herein in which full length A box polypeptides are used, for example, inhibiting release of a proinflammatory cytokine from a cell, or treating a patient having a condition characterized by activation of an inflammatory cytokine cascade.

As used herein, an “HMGB B box”, also referred to herein as a “B box”, is a substantially pure, or substantially pure and isolated polypeptide, that has been separated from components that naturally accompany it, and consists of an amino acid sequence that is less than a full length HMGB polypeptide and has one or more of the following biological activities: increasing inflammation, increasing release of a proinflammatory cytokine from a cell, and or increasing the activity of the inflammatory cytokine cascade. In one embodiment, the HMGB B box polypeptide has one of the above biological activities. In another embodiment, the HMGB B box polypeptide has two of the above biological activities. In a third embodiment, the HMGB B box polypeptide has all three of the above biological activities. Preferably, the HMGB B box has at least 25%, 30%, 40%, 50%, 60%, 70%, 80% or 90% of the biological activity of a full length HMGB polypeptide. In another embodiment, the HMGB B box does not comprise an HMGB A box.

In another embodiment, the HMGB B box is a polypeptide that is about 90%, 80%, 70%, 60%, 50%, 40%, 35%, 30%, 25%, or 20%, of the length of a full length HMGB1 polypeptide. In another embodiment, the HMGB box comprises or consists of the sequence of SEQ ID NO:5, SEQ ID NO:20, or SEQ ID NO:58, or the amino acid sequence in the corresponding region of an HMGB protein in a mammal, but is still less than the full length HMGB polypeptide. An HMGB B box polypeptide is also an artificially or recombinantly produced polypeptide having the same amino acid sequence as an HMGB B box polypeptide described above. Preferably, the HMGB B box is a mammalian HMGB B box, for example, a human HMGB1 B box. An HMGB B box often has no more than about 85 amino acids and no fewer than about 4 amino acids. Examples of polypeptides having B box sequences within them include, but are not limited to HMGB polypeptides described herein. The B box sequences in such polypeptides can be determined and isolated using methods described herein, for example, by sequence comparisons to B boxes described herein and testing for biological activity, using methods described herein or other methods known in the art.

Examples of HMGB B box polypeptide sequences include the following sequences: FKDPNAPKRP PSAFFLFCSE YRPKIKGEHP GLSIGDVAKK LGEMWNNTAA DDKQPYEKKA AKLKEKYEKD IAAY (human HMGB1; SEQ ID NO: 51); KKDPNAPKRP PSAFFLFCSE HRPKIKSEHP GLSIGDTAKK LGEMWSEQSA KDKQPYEQKA AKLKEKYEKD IAAY (human HMGB2; SEQ ID NO: 52); FKDPNAPKRL PSAFFLFCSE YRPKIKGEHP GLSIGDVAKK LGEMWNNTAA DDKQPYEKKA AKLKEKYEKD IAAY (HMG1L5 (formerly HMG1L10); SEQ ID NO: 53); FKDPNAPKRP PSAFFLFCSE YHPKIKGEHP GLSIGDVAKK LGEMWNNTAA DDKQPGEKKA AKLKEKYEKD IAAY (HMG1L1; SEQ ID NO: 54); FKDSNAPKRP PSAFLLFCSE YCPKIKGEHP GLPISDVAKK LVEMWNNTFA DDKQLCEKKA AKLKEKYKKD TATY (HMG1L4; SEQ ID NO: 55); FKDPNAPKRP PSAFFLFCSE YRPKIKGEHP GLSIGDVVKK LAGMWNNTAA ADKQFYEKKA AKLKEKYKKD IAAY (HMG sequence from BAC clone RP11-359A23; SEQ ID NO: 56); and FKDPNAPKRP PSAFFLFCSE YRPKIKGEHP GLSIGDVAKK LGEMWNNTAA DDKQPYEKKA AKLKEKYEKD IAAYRAKGKP DAAKKGVVKA EK (human HMGB1 box; SEQ ID NO: 58).

The HMGB B box polypeptides of the invention also encompass sequence variants. Variants include a substantially homologous polypeptide encoded by the same genetic locus in an organism, i.e., an allelic variant, as well as other variants. Variants also encompass polypeptides derived from other genetic loci in an organism, but having substantial homology to a polypeptide encoded by an HMGB nucleic acid molecule, and complements and portions thereof, or having substantial homology to a polypeptide encoded by a nucleic acid molecule comprising the nucleotide sequence of an HMGB B box nucleic acid molecule. Examples of HMGB B box nucleic acid molecules are known in the art and can be derived from HMGB B box polypeptides as described herein. Variants also include polypeptides substantially homologous or identical to these polypeptides but derived from another organism, i.e., an ortholog. Variants also include polypeptides that are substantially homologous or identical to these polypeptides that are produced by chemical synthesis. Variants also include polypeptides that are substantially homologous or identical to these polypeptides that are produced by recombinant methods. Preferably, a non-naturally occurring HMGB B box polypeptide has at least 60%, more preferably, at least 70%, 75%, 80%, 85%, or 90%, and most preferably at least 95% sequence identity to the sequence of an HMGB B box as described herein, for example, the sequence of SEQ ID NO:5, SEQ ID NO:20, or SEQ ID NO:58, as determined using the BLAST program and parameters described herein. Preferably, the HMGB B box consists of the sequence of SEQ ID NO:5, SEQ ID NO:20, or SEQ ID NO:58, or the amino acid sequence in the corresponding region of an HMGB protein in a mammal.

In other embodiments, the present invention is directed to a polypeptide comprising an HMGB B box biologically active fragment that has B box biological activity, or a non-naturally occurring HMGB B box fragment By a “B box fragment that has B box biological activity” or a “B box biologically active fragment” is meant a fragment of an HMGB B box that has the activity of an HMGB B box. For example, the B box fragment can induce release of a pro-inflammatory cytokine from a vertebrate cell or increase inflammation, or induce the inflammatory cytokine cascade. An example of such a B box fragment is the fragment comprising the first 20 amino acids of the HMGB1 B box (SEQ ID NO:16 or SEQ ID NO:23), as described herein. B box fragments can be generated using standard molecular biology techniques and assaying the function of the fragment by determining if the fragment, when administered to a cell, increases release of a proinflammatory cytokine from the cell, as compared to a suitable control, for example, using methods described herein.

HMGB polypeptides, HMGB A boxes, and HMGB B boxes, either naturally occurring or non-naturally occurring, include polypeptides that have sequence identity to the HMGB polypeptides, HMGB A boxes, and HMGB B boxes described herein. As used herein, two polypeptides (or a region of the polypeptides) are substantially homologous or identical when the amino acid sequences are at least about 60%, 70%, 75%, 80%, 85%, 90% or 95% or more homologous or identical. The percent identity of two amino acid sequences (or two nucleic acid sequences) can be determined by aligning the sequences for optimal comparison purposes (e.g., gaps can be introduced in the sequence of a first sequence). The amino acids or nucleotides at corresponding positions are then compared, and the percent identity between the two sequences is a function of the number of identical positions shared by the sequences (i.e., % identity=# of identical positions/total # of positions×100). In certain embodiments, the length of the HMGB polypeptide, HMGB A box polypeptide, or HMGB B box polypeptide aligned for comparison purposes is at least 30%, preferably, at least 40%, more preferably, at least 60%, and even more preferably, at least 70%, 80%, 90%, or 100%, of the length of the reference sequence, for example, those sequence provided in FIGS. 12A-12E, FIGS. 14A-14P, and SEQ ID NOS: 18, 20, and 22. The actual comparison of the two sequences can be accomplished by well-known methods, for example, using a mathematical algorithm. A preferred, non-limiting example of such a mathematical algorithm is described in Karlin et al. (Proc. Natl. Acad. Sci. USA, 90:5873-5877, 1993). Such an algorithm is incorporated into the BLASTN and BLASTX programs (version 2.2) as described in Schaffer et al., (Nucleic Acids Res., 29:2994-3005, 2001). When utilizing BLAST and Gapped BLAST programs, the default parameters of the respective programs (e.g., BLASTN; available at the Internet site for the National Center for Biotechnology Information) can be used. In one embodiment, the database searched is a non-redundant (NR) database, and parameters for sequence comparison can be set at: no filters; Expect value of 10; Word Size of 3; the Matrix is BLOSUM62; and Gap Costs have an Existence of 11 and an Extension of 1.

Another preferred, non-limiting example of a mathematical algorithm utilized for the comparison of sequences is the algorithm of Myers and Miller, CABIOS (1989). Such an algorithm is incorporated into the ALIGN program (version 2.0), which is part of the GCG (Accelrys, San Diego, Calif.) sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (Comput. Appl. Biosci., 10: 3-5, 1994); and FASTA described in Pearson and Lipman (Proc. Natl. Acad. Sci USA, 85: 2444-2448, 1988).

In another embodiment, the percent identity between two amino acid sequences can be accomplished using the GAP program in the GCG software package (Accelrys, San Diego, Calif.) using either a Blossom 63 matrix or a PAM250 matrix, and a gap weight of 12, 10, 8, 6, or 4 and a length weight of 2, 3, or 4. In yet another embodiment, the percent identity between two nucleic acid sequences can be accomplished using the GAP program in the GCG software package (Accelrys, San Diego, Calif.), using a gap weight of 50 and a length weight of 3.

As used herein, a “cytokine” is a soluble protein or peptide which is naturally produced by mammalian cells and which acts in vivo as a humoral regulator at micro- to picomolar concentrations. Cytokines can, either under normal or pathological conditions, modulate the functional activities of individual cells and tissues. A proinflammatory cytokine is a cytokine that is capable of causing any of the following physiological reactions associated with inflammation: vasodilation, hyperemia, increased permeability of vessels with associated edema, accumulation of granulocytes and mononuclear phagocytes, and deposition of fibrin. In some cases, the proinflammatory cytokine can also cause apoptosis, such as in chronic heart failure, where TNF has been shown to stimulate cardiomyocyte apoptosis (Pulkki, Ann. Med. 29:339-343, 1997; and Tsutsui et al., Immunol. Rev. 174:192-209, 2000).

Nonlimiting examples of proinflammatory cytokines are tumor necrosis factor (TNF), interleukin (IL)-1α, IL-1β, IL-6, IL-8, IL-18, interferon γ, HMG-1, platelet-activating factor (PAF) and macrophage migration inhibitory factor (MIF).

Proinflammatory cytokines are to be distinguished from anti-inflammatory cytokines, such as IL-4, IL-10, and IL-13, which are not mediators of inflammation.

In many instances, proinflammatory cytokines are produced in an inflammatory cytokine cascade, defined herein as an in vivo release of at least one proinflammatory cytokine in a mammal, wherein the cytokine release affects a physiological condition of the mammal. Thus, an inflammatory cytokine cascade is inhibited in embodiments of the invention where proinflammatory cytokine release causes a deleterious physiological condition.

As used herein, “an agent that inhibits TNF biological activity” is an agent that decreases one or more of the biological activities of TNF. Examples of TNF biological activity include, but are not limited to, vasodilation, hyperemia, increased permeability of vessels with associated edema, accumulation of granulocytes and mononuclear phagocytes, and deposition of fibrin. Agents that inhibit TNF biological activity include agents that inhibit (decrease) the interaction between TNF and a TNF receptor. Examples of such agents include antibodies or antigen binding fragments thereof that bind to TNF, antibodies or antigen binding fragments that bind a TNF receptor, and molecules that bind TNF or the TNF receptor and prevent TNF/TNF receptor interaction. Such agents include, but are not limited to peptides, proteins, synthesized molecules, for example, synthetic organic molecules, naturally-occurring molecule, for example, naturally occurring organic molecules, nucleic acid molecules, and components thereof. Preferred examples of agents that inhibit TNF biological activity include infliximab (Remicade; Centocor, Inc., Malvern, Pa.), etanercept (Immunex; Seattle, Wash.), adalimumab (D2E7; Abbot Laboratories, Abbot Park Ill.), CDP870 (Pharmacia Corporation; Bridgewater, N.J.) CDP571 (Celltech Group plc, United Kingdom), Lenercept (Roche, Switzerland), and Thalidomide.

Inflammatory cytokine cascades contribute to deleterious characteristics, including inflammation and apoptosis, of numerous disorders. Included are disorders characterized by both localized and systemic reactions, including, without limitation, sepsis, allograft rejection, rheumatoid arthritis, asthma, lupus, adult respiratory distress syndrome, chronic obstructive pulmonary disease, psoriasis, pancreatitis, peritonitis, burns, myocardial ischemia, organic ischemia, reperfusion ischemia, Behcet's disease, graft versus host disease, Crohn's disease, ulcerative colitis, multiple sclerosis, and cachexia.

A Box Polypeptides and Biologically Active Fragments Thereof

As described above, the present invention is directed to compositions comprising an HMGB A box, or a biologically active fragment or variant thereof, in combination with one or more agents that inhibit TNF biological activity, for example, infliximab, etanercept, adalimumab, CDP870, CDP571, Lenercept, or Thalidomide. Such compositions can be used to inhibit release of a proinflammatory cytokine from a vertebrate cell treated with HMG, and/or can be used to treat a condition characterized by activation of an inflammatory cytokine cascade.

When referring to the effect of any of the compositions or methods of the invention on the release of proinflammatory cytokines, the use of the terms “inhibit” or “decrease” encompasses at least a small but measurable reduction in proinflammatory cytokine release. In preferred embodiments, the release of the proinflammatory cytokine is inhibited by at least 10%, 20%, 25%, 30%, 40%, 50%, 75%, 80%, or 90% over non-treated controls. Inhibition can be assessed using methods described herein or other methods known in the art. Such reductions in proinflammatory cytokine release are capable of reducing the deleterious effects of an inflammatory cytokine cascade in in vivo embodiments.

Because HMGB A boxes show a high degree of sequence conservation (see, for example, FIG. 13 for an amino acid sequence comparison of rat, mouse, and human HMGB polypeptides), it is reasonable to believe that HMGB A boxes generally can inhibit release of a proinflammatory cytokine from a vertebrate cell treated with an HMGB polypeptide. Preferably, the HMGB A box is a vertebrate HMGB A box, for example, a mammalian HMGB A box (e.g., a mammalian HMGB1 A box, such as a human HMGB1 A box provided herein as SEQ ID NO:4 or SEQ ID NO:22 or SEQ ID NO:57). Also included in the present invention are fragments of the HMGB1 A box having HMGB A box biological activity, as described herein.

It would also be recognized by the skilled artisan that non-naturally occurring HMGB A boxes (or biologically active fragments thereof) can be created without undue experimentation, which would inhibit release of a proinflammatory cytokine from a vertebrate cell treated with an HMGB polypeptide. These non-naturally occurring functional A boxes (variants) can be created by aligning amino acid sequences of HMGB A boxes from different sources, and making one or more substitutions in one of the sequences at amino acid positions where the A boxes differ. The substitutions are preferably made using the same amino acid residue that occurs in the compared A box. Alternatively, a conservative substitution is made from either of the residues.

Conservative amino acid substitutions refer to the interchangeability of residues having similar side chains. Conservatively substituted amino acids can be grouped according to the chemical properties of their side chains. For example, one grouping of amino acids includes those amino acids have neutral and hydrophobic side chains (a, v, l, i, p, w, f, and m); another grouping is those amino acids having neutral and polar side chains (g, s, t, y, c, n, and q); another grouping is those amino acids having basic side chains (k, r, and h); another grouping is those amino acids having acidic side chains (d and e); another grouping is those amino acids having aliphatic side chains (g, a, v, l, and i); another grouping is those amino acids having aliphatic-hydroxyl side chains (s and t); another grouping is those amino acids having amine-containing side chains (n, q, k, r, and h); another grouping is those amino acids having aromatic side chains (f, y, and w); and another grouping is those amino acids having sulfur-containing side chains (c and m). Preferred conservative amino acid substitutions groups are: r-k; e-d, y-f, 1-m; v-i, and q-h.

While a conservative amino acid substitution would be expected to preserve the biological activity of an HMGB A box polypeptide, the following is one example of how non-naturally occurring A box polypeptides can be made by comparing the human HMGB1 A box (SEQ ID NO:4) with residues 32 to 85 of SEQ ID NO:3 of the human HMGB2 A box (SEQ ID NO:17). HMGB1 pdasvnfsef skkcserwkt msakekgkfe dmakadkary eremktyipp kget (SEQ ID NO:4) HMGB2 pdssvnfaef skkcserwkt msakekskfe dmaksdkary dremknyvpp kgdk (SEQ ID NO:17)

A non-naturally occurring HMGB A box can be created by, for example, by substituting the alanine (a) residue at the third position in the HMGB1 A box with the serine (s) residue that occurs at the third position of the HMGB2 A box. The skilled artisan would know that the substitution would provide a functional non-naturally occurring A box because the s residue functions at that position in the HMGB2 A box. Alternatively, the third position of the HMGB1 A box can be substituted with any amino acid that is conservative to alanine or serine, such as glycine (g), threonine (t), valine (v) or leucine (l). The skilled artisan would recognize that these conservative substitutions would be expected to result in a functional A box because A boxes are not invariant at the third position, so a conservative substitution would provide an adequate structural substitute for an amino acid that is naturally occurring at that position.

Following the above method, a great many non-naturally occurring HMGB A boxes could be created without undue experimentation which would be expected to be functional, and the functionality of any particular non-naturally occurring HMGB A box could be predicted with adequate accuracy. In any event, the functionality of any non-naturally occurring HMGB A box could be determined without undue experimentation by simply adding it to cells along with an HMG polypeptide, and determining whether the A box inhibits release of a proinflammatory cytokine by the cells, using, for example, methods described herein.

The cell from which the A box or an A box biologically active fragment will inhibit the release of HMG-induced proinflammatory cytokines can be any cell that can be induced to produce a proinflammatory cytokine. In preferred embodiments, the cell is a mammalian cell, for example, an immune cell (e.g., a macrophage, a monocyte, or a neutrophil).

B Box Polypeptides, and Biologically Active Fragments Thereof

As described herein, a polypeptide composition comprising a vertebrate HMGB B box, or a biologically active fragment thereof can be used to increase release of a proinflammatory cytokine from a vertebrate cell treated with HMGB.

When referring to the effect of any of the compositions or methods of the invention on the release of proinflammatory cytokines, the use of the term “increase” encompasses at least a small but measurable rise in proinflammatory cytokine release. In preferred embodiments, the release of the proinflammatory cytokine is increased by at least 1.5-fold, at least 2-fold, at least 5-fold, or at least 10-fold, over non-treated controls. Such increases in proinflammatory cytokine release are capable of increasing the effects of an inflammatory cytokine cascade in in vivo embodiments.

Because all HMGB B boxes show a high degree of sequence conservation (see, for example, FIG. 13 for an amino acids sequence comparison of rat, mouse, and human HMGB polypeptides), it is believed that functional non-naturally occurring HMGB B boxes (variants) can be created without undue experimentation by making one or more conservative amino acid substitutions, or by comparing naturally occurring vertebrate B boxes from different sources and substituting analogous amino acids, as was discussed above with respect to the creation of functional non-naturally occurring A boxes. In particularly preferred embodiments, the B box comprises SEQ ID NO:5, SEQ ID NO:20, or SEQ ID NO:58, which are the sequences (three different lengths) of the human HMGB1 B box, or is a fragment of an HMGB B box that has B box biological activity. For example, a 20 amino acid sequence contained within SEQ ID NO:20 contributes to the function of the B box. This 20 amino acid B-box fragment has the following amino acid sequence: fkdpnapkrl psafflfcse (SEQ ID NO:23). Another example of an HMGB B box biologically active fragment consists of amino acids 1-20 of SEQ ID NO:5 (napkrppsaf flfcseyrpk; SEQ ID NO:16).

Antibodies to HMGB and HMGB B Box Polypeptides

The invention is also directed to a purified preparation of antibodies that bind to an HMGB polypeptide or a biologically active fragment thereof (anti-HMGB antibodies). The anti-HMGB antibodies can be neutralizing antibodies (i.e., can inhibit a biological activity of an HMG polypeptide or a biologically active fragment thereof, for example, the release of a proinflammatory cytokine from a vertebrate cell induced by HMG). The invention also features antibodies that selectively bind to a vertebrate high mobility group protein (HMG) B box or a biologically active fragment thereof, but do not selectively bind to non-B box epitopes of HMGB (anti-HMGB B box antibodies). In this embodiment, the antibodies can also be neutralizing antibodies (i.e., they can inhibit a biological activity of a B box polypeptide or biologically active fragment thereof, for example, the release of a proinflammatory cytokine from a vertebrate cell induced by HMGB). Such antibodies can be combined with one or more agents that inhibit TNF biological activity, for example, infliximab, etanercept, adalimumab, CDP870, CDP571, Lenercept, or Thalidomide.

The term “antibody” or “purified antibody” as used herein refers to immunoglobulin molecules and immunologically active portions of immunoglobulin molecules, i.e., molecules that contain an antigen binding site that selectively binds an antigen (antigen binding fragments). A molecule that selectively binds to a polypeptide of the invention is a molecule that binds to that polypeptide or a fragment thereof, but does not substantially bind other molecules in a sample, e.g., a biological sample that naturally contains the polypeptide. Preferably the antibody is at least 60%, by weight, free from proteins and naturally occurring organic molecules with which it is naturally associated. More preferably, the antibody preparation is at least 75% or 90%, and most preferably, 99%, by weight, antibody. Examples of immunologically active portions of immunoglobulin molecules include F(v), F(ab), F(ab′) and F(ab′)₂ fragments that can be generated by treating the antibody with an enzyme such as pepsin.

The invention provides polyclonal and monoclonal antibodies that selectively bind to a HMGB B box polypeptide of the invention. The term “monoclonal antibody” or “monoclonal antibody composition,” as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared, e.g., as described herein, by immunizing a suitable subject with a desired immunogen, e.g., an HMGB polypeptide, an HMGB B box polypeptide, or fragments thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985) or trioma techniques. The technology for producing hybridomas is well known (see generally Current Protocols in Immunology, Coligan et al., (eds.) John Wiley & Sons, Inc., New York, N.Y., 1994). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a particular polypeptide, e.g., a polypeptide described herein.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al., Nature, 266:55052, 1977; R. H. Kenneth, in Monoclonal Antibodies: A New Dimension In Biological Analyses, Plenum Publishing Corp., New York, N.Y., 1980; and Lerner, Yale J. Biol. Med. 54:387-402, 1981). Moreover, the ordinarily skilled worker will appreciate that there are many variations of such methods that also would be useful.

In one alternative to preparing monoclonal antibody-secreting hybridomas, a monoclonal antibody to an HMGB polypeptide or an HMGB B box polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al., Bio/Technology 9:1370-1372, 1991; Hay et al., Hum. Antibod. Hybridomas 3:81-85, 1992; Huse et al., Science 246:1275-1281,1989; and Griffiths et al., EMBO J. 12:725-734, 1993.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

Because vertebrate HMGB polypeptides and HMGB B boxes show a high degree of sequence conservation, it is reasonable to believe that vertebrate HMGB polypeptides or HMGB B boxes in general can induce release of a proinflammatory cytokine from a vertebrate cell. Therefore, antibodies against vertebrate HMGB polypeptides or HMGB B boxes without limitation are within the scope of the invention. In one embodiment, the antibodies are neutralizing antibodies.

Preferably, the HMGB polypeptide is a mammalian HMG, as described herein, more preferably a mammalian HMGB1 polypeptide, most preferably a human HMGB1 polypeptide, provided herein as SEQ ID NO:1. Antibodies can also be directed against an HMGB polypeptide fragment that has HMGB polypeptide biological activity.

Preferably, the HMGB B box is a mammalian HMGB B box, more preferably a mammalian HMGB1 B box, most preferably a human HMGB1 B box, provided herein as SEQ ID NO:5, SEQ ID NO:20, or SEQ ID NO:58. Antibodies can also be directed against an HMGB B box fragment that has B box biological activity.

Antibodies generated against an HMGB immunogen or an HMGB B box immunogen can be obtained by administering an HMGB polypeptide, or fragment thereof, an HMGB B box or fragment thereof, or cells comprising the HMGB polypeptide, the HMGB B box, or fragments thereof, to an animal, preferably a nonhuman, using routine protocols. The polypeptide, such as an antigenically or immunologically equivalent derivative, is used as an antigen to immunize a mouse or other animal, such as a rat or chicken. The immunogen may be associated, for example, by conjugation, with an immunogenic carrier protein, for example, bovine serum albumin (BSA) or keyhole limpet haemocyanin (KLH). Alternatively, a multiple antigenic peptide comprising multiple copies of the HMGB or HMGB B box or fragment, may be sufficiently antigenic to improve immunogenicity so as to obviate the need for a carrier. Bispecific antibodies, having two antigen binding domains where each is directed against a different HMGB or HMGB B box epitope, may also be produced by routine methods.

For preparation of monoclonal antibodies, any technique known in the art that provides antibodies produced by continuous cell line cultures can be used. See, e.g., Kohler and Milstein, Nature 256: 495-497, 1975; Kozbor et al., Immunology Today 4:72, 1983; and Cole et al., pp. 77-96 in MONOCLONAL ANTIBODIES AND CANCER THERAPY, Alan R. Liss, Inc., 1985.

Techniques for the production of single chain antibodies (U.S. Pat. No. 4,946,778) can be adapted to produce single chain antibodies to the HMGB polypeptides or HMGB B box polypeptides or fragments thereof. Also, transgenic mice, or other organisms such as other mammals, may be used to express humanized antibodies.

If the antibody is used therapeutically in in vivo applications, the antibody is preferably modified to make it less immunogenic in the individual. For example, if the individual is human the antibody is preferably “humanized”; where the complementarity determining region(s) of the antibody is transplanted into a human antibody (for example, as described in Jones et al., Nature 321:522-525, 1986; and Tempest et al., Biotechnology 9:266-273, 1991).

Phage display technology can also be utilized to select antibody genes with binding activities towards the polypeptide either from repertoires of PCR amplified v-genes of lymphocytes from humans screened for possessing anti-B box antibodies or from naive libraries (McCafferty et al., Nature 348:552-554, 1990; and Marks, et al., Biotechnology 10:779-783, 1992). The affinity of these antibodies can also be improved by chain shuffling (Clackson et al., Nature 352: 624-628, 1991).

When the antibodies are obtained that specifically bind to HMGB epitopes or to HMGB B box epitopes, they can then be screened, without undue experimentation, for the ability to inhibit release of a proinflammatory cytokine. Anti-HMGB antibodies and anti-HMGB B box antibodies that can inhibit the production of any single proinflammatory cytokine and/or the release of a proinflammatory cytokine from a cell, and/or inhibit a condition characterized by activation of an inflammatory cytokine cascade are within the scope of the present invention. Preferably, the antibodies can inhibit the production of TNF, IL-1β, and/or IL-6.

Compositions Comprising an HMGB A box polypeptide, an Antibody to HMGB, Antibodies to an HMGB B box, and an Inhibitor of TNF Biological Activity

The present invention is also directed to a composition comprising any of the above-described HMGB A box polypeptides, and/or an antibody or antigen binding fragment thereof that binds HMGB, and/or an antibody or antigen binding fragment thereof that binds an HMGB B box, and an agent that inhibits TNF biological activity (collectively termed “combination therapy compositions”). Preferred examples of agents that inhibit TNF biological activity include infliximab, etanercept, adalimumab, CDP870, CDP571, Lenercept, and Thalidomide. Such combination therapy compositions can further comprise a pharmaceutically acceptable carrier. In these embodiments, the combination therapy composition can inhibit a condition characterized by activation of an inflammatory cytokine cascade and/or inhibit release of a proinflammatory cytokine from a cell. The condition can be one where the inflammatory cytokine cascade causes a systemic reaction, such as with endotoxic shock. Alternatively, the condition can be mediated by a localized inflammatory cytokine cascade, as in rheumatoid arthritis. Nonlimiting examples of conditions which can be usefully treated using the present invention include sepsis, allograft rejection, rheumatoid arthritis, asthma, lupus, adult respiratory distress syndrome, chronic obstructive pulmonary disease, psoriasis, pancreatitis, peritonitis, burns, myocardial ischemia, organic ischemia, reperfusion ischemia, Behcet's disease, graft versus host disease, Crohn's disease, ulcerative colitis, multiple sclerosis, and cachexia. Preferably the combination therapy compositions are administered to a patient in need thereof in an amount sufficient to inhibit release of proinflammatory cytokine from a cell and/or to treat a condition characterized by activation of an inflammatory cytokine cascade. In one embodiment, release of the proinflammatory cytokine is inhibited by at least 10%, 20%, 25%, 50%, 75%, 80%, 90% or 95%, as assessed using methods described herein or other methods known in the art.

The carrier included with the combination therapy compositions is chosen based on the expected route of administration of the composition in therapeutic applications. The route of administration of the composition depends on the condition to be treated. For example, intravenous injection may be preferred for treatment of a systemic disorder such as endotoxic shock, and oral administration may be preferred to treat a gastrointestinal disorder such as a gastric ulcer. The route of administration and the dosage of the combination therapy composition to be administered can be determined by the skilled artisan, without undue experimentation, in conjunction with standard dose-response studies. Relevant circumstances to be considered in making such determinations include the condition or conditions to be treated, the age, weight, and response of the individual patient, and the severity of the patient's symptoms. Thus, depending on the condition, the combination therapy composition can be administered orally, parenterally, intranasally, vaginally, rectally, lingually, sublingually, bucally, intrabuccaly and/or transdermally to the patient.

Accordingly, combination therapy compositions designed for oral, lingual, sublingual, buccal and intrabuccal administration can be made without undue experimentation by means well known in the art, for example, with an inert diluent or with an edible carrier. The combination therapy composition may be enclosed in gelatin capsules or compressed into tablets. For the purpose of oral therapeutic administration, the pharmaceutical compositions of the present invention may be incorporated with excipients and used in the form of tablets, troches, capsules, elixirs, suspensions, syrups, wafers, chewing gums and the like.

Tablets, pills, capsules, troches and the like may also contain binders, recipients, disintegrating agent, lubricants, sweetening agents, and/or flavoring agents. Some examples of binders include microcrystalline cellulose, gum tragacanth and gelatin. Examples of excipients include starch and lactose. Some examples of disintegrating agents include alginic acid, corn starch and the like. Examples of lubricants include magnesium stearate and potassium stearate. An example of a glidant is colloidal silicon dioxide. Some examples of sweetening agents include sucrose, saccharin and the like. Examples of flavoring agents include peppermint, methyl salicylate, orange flavoring and the like. Materials used in preparing these various compositions should be pharmaceutically pure and non-toxic in the amounts used.

The combination therapy compositions of the present invention can easily be administered parenterally such as, for example, by intravenous, intramuscular, intrathecal or subcutaneous injection. Parenteral administration can be accomplished by incorporating the combination therapy compositions of the present invention into a solution or suspension. Such solutions or suspensions may also include sterile diluents such as water for injection, saline solution, fixed oils, polyethylene glycols, glycerine, propylene glycol and/or other synthetic solvents. Parenteral formulations may also include antibacterial agents such as, for example, benzyl alcohol and/or methyl parabens, antioxidants such as, for example, ascorbic acid and/or sodium bisulfite, and chelating agents, such as EDTA. Buffers, such as acetates, citrates and/or phosphates and agents for the adjustment of tonicity, such as sodium chloride and/or dextrose, may also be added. The parenteral preparation can be enclosed in ampules, disposable syringes or multiple dose vials made of glass or plastic.

Rectal administration includes administering the combination therapy composition into the rectum or large intestine. This can be accomplished using suppositories or enemas. Suppository formulations can easily be made by methods known in the art. For example, suppository formulations can be prepared by heating glycerin to about 120° C., dissolving the pharmaceutical composition in the glycerin, mixing the heated glycerin after which purified water may be added, and pouring the hot mixture into a suppository mold.

Transdermal administration includes percutaneous absorption of the composition through the skin. Transdermal formulations include patches, ointments, creams, gels, salves and the like.

The present invention includes nasally administering to a patient a therapeutically effective amount of the combination therapy composition. As used herein, nasally administering or nasal administration includes administering the combination therapy compositions to the mucous membranes of the nasal passage or nasal cavity of the patient. As used herein, pharmaceutical compositions for nasal administration of a composition include therapeutically effective amounts of the combination therapy composition prepared by well-known methods, to be administered, for example, as a nasal spray, nasal drop, suspension, gel, ointment, cream or powder. Administration of the composition may also take place using a nasal tampon or nasal sponge.

If desired, the combination therapy compositions described herein can also include an antagonist of an early sepsis mediator. As used herein, an early sepsis mediator is a proinflammatory cytokine that is released from cells soon (i.e., within 30-60 min.) after induction of an inflammatory cytokine cascade (e.g., exposure to LPS). Nonlimiting examples of these cytokines are IL-1α, IL-11β, IL-6, PAF, and MIF. Also included as early sepsis mediators are receptors for these cytokines (for example, tumor necrosis factor receptor type 1) and enzymes required for production of these cytokines, for example, interleukin-1β converting enzyme). Antagonists of any early sepsis mediator, now known or later discovered, can be useful for these embodiments by further inhibiting an inflammatory cytokine cascade.

Nonlimiting examples of antagonists of early sepsis mediators are antisense compounds that bind to the mRNA of the early sepsis mediator, preventing its expression (see, e.g., Ojwang et al., Biochemistry 36:6033-6045, 1997; Pampfer et al., Biol. Reprod. 52:1316-1326, 1995; U.S. Pat. No. 6,228,642; Yahata et al., Antisense Nucleic Acid Drug Dev. 6:55-61, 1996; and Taylor et al., Antisense Nucleic Acid Drug Dev. 8:199-205, 1998), ribozymes that specifically cleave the mRNA of the early sepsis mediator (see, e.g., Leavitt et al., Antisense Nucleic Acid Drug Dev. 10: 409-414, 2000; Kisich et al., 1999; and Hendrix et al., Biochem. J. 314 (Pt. 2): 655-661, 1996), and antibodies that bind to the early sepsis mediator and inhibit their action (see, e.g., Kam and Targan, Expert Opin. Pharmacother. 1: 615-622, 2000; Nagahira et al., J. Immunol. Methods 222, 83-92, 1999; Lavine et al., J. Cereb. Blood Flow Metab. 18: 52-58, 1998; and Holmes et al., Hybridoma 19: 363-367, 2000). Any antagonist of an early sepsis mediator, now known or later discovered, is envisioned as within the scope of the invention. The skilled artisan can determine the amount of early sepsis mediator to use in these compositions for inhibiting any particular inflammatory cytokine cascade without undue experimentation with routine dose-response studies.

Other agents that can be administered with the combination therapy compositions described herein include, e.g., Vitaxin™^{m} and other antibodies targeting αvβ3 integrin (see, e.g., U.S. Pat. No. 5,753,230, PCT Publication Nos. WO 00/78815 and WO 02/070007; the entire teachings of all of which are incorporated herein by reference) and anti-IL-9 antibodies (see, e.g., PCT Publication No. WO 97/08321; the entire teachings of which are incorporated herein by reference). Additional agents that can be administered with the polypeptide compositions described herein include, e.g., B7 antagonists (e.g., CTLA4Ig, anti-CD80 antibodies, anti-CD86 antibodies), methotrexate, and/or CD40 antagonists (e.g., anti-CD40 ligand (CD40L)) (see, e.g., Saito et al., J. Immunol. 160(9):4225-31 (1998)).

Preferred embodiments of the invention are described in the following examples. Other embodiments within the scope of the invention will be apparent to one skilled in the art from consideration of the specification or practice of the invention as disclosed herein. It is intended that the specification, together with the examples and claims, be considered exemplary only.

### EXAMPLE 1

### Materials and Methods

Cloning of HMGB1 and Production of HMGB1 Mutants

The following methods were used to prepare clones and mutants of human HMGB1. Recombinant full length human HMGB1 (651 base pairs; GenBank Accession Number U51677) was cloned by PCR amplification from a human brain Quick-Clone cDNA preparation (Clontech, Palo Alto, Calif.) using the following primers; forward primer: 5′ GATGGGCAAAGGAGATCCTAAG 3′ (SEQ ID NO:6) and reverse primer: 5′ GCGGCCGCTTATTCATCATCATCATCTTC 3′ (SEQ ID NO:7). Human HMGB1 mutants were cloned and purified as follows. A truncated form of human HMGB1 was cloned by PCR amplification from a Human Brain Quick-Clone cDNA preparation (Clontech, Palo Alto, Calif.). The primers used were (forward and reverse, respectively):

Carboxy terminus mutant (557 bp): 5′ GATGGGCAAAGGAGATCCTAAG 3′ (SEQ ID NO:8) and 5′ GCGGCCGC TCACTTGCTTTTTTCAGCCTTGAC 3′ (SEQ ID NO:9).

Amino terminus+B box mutant (486 bp): 5′ GAGCATAAGAAGAAGCACCCA 3′ (SEQ ID NO:10) and 5′ GCGGCCGC TCACTTGCTTTTTTCAGCCTTGAC 3′ (SEQ ID NO:11).

B box mutant (233 bp): 5′ AAGTTCAAGGATCCCAATGCAAAG 3′ (SEQ ID NO:12) and 5′ GCGGCCGCTCAATATGCAGCTATATCCTTTTC 3′ (SEQ ID NO:13).

Amino terminus+A box mutant (261 bp): 5′ GATGGGCAAAGGAGATCCTAAG 3′ (SEQ ID NO:14) and 5′ TCACTTTTTTGTCTCCCCTTTGGG 3′ (SEQ ID NO:15).

A stop codon was added to each mutant to ensure the accuracy of protein size. PCR products were subcloned into pCRII-TOPO vector EcoRI sites using the TA cloning method per manufacturer's instruction (Invitrogen, Carlsbad, Calif.). After amplification, the PCR product was digested with EcoRI and subcloned into an expression vector with a GST tag pGEX (Pharmacia); correct orientation and positive clones were confirmed by DNA sequencing on both strands. The recombinant plasmids were transformed into protease deficient E. Coli strains BL21 or BL21 (DE3)plysS (Novagen, Madison, Wis.) and fusion protein expression was induced by isopropyl-D-thiogalactopyranoside (IPTG). Recombinant proteins were obtained using affinity purification with the glutathione Sepharose resin column (Pharmacia).

The HMGB mutants generated as described above have the following amino acid sequences:

Wild Type HMGB1:

| | |
|---|---|
| MGKGDPKKPTGKMSSYAFFVQTCREEHKKKHPDASVNFSEFSKKCSERWKTM | (SEQ ID NO: 18) |
| SAKEKGKFEDMAKADKARYEREMKTYIPPKGETKKKFKDPNAPKRLPSAFFLF | |
| CSEYRPKIKGEHPGLSIGDVAKKLGEMWNNTAADDKQPYEKKAAKLKEKYEK | |
| DIAAYRAKGKPDAAKKGVVKAEKSKKKKEEEEDEEDEEDEEEEEDEEDEEDEE | |
| EDDDDE | |

Carboxy terminus mutant: MGKGDPKKPTGKMSSYAFFVQTCREEHKKKHPDAS VNFSEFSKKCSERWKTMSAKEKGKFEDMAKADKARYEREMKTYIPPKGETKK KFKDPNAPKRLPSAFFLFCSEYRPKIKGEHPGLSIGDVAKKLGEMWNNTAADDK QPYEKKAAKLKEKYEKDIAAYRAKGKPDAAKKGVVKAEKSK (SEQ ID NO:19)

B Box mutant: FKDPNAPKRLPSAFFLFCSEYRPKIKGEHPGLSIGDVAKKLGEM WNNTAADDKQPYEKKAAKLKEKYEKDIAAY (SEQ ID NO:20)

Amino terminus+A Box mutant: MGKGDPKKPTGKMSSYAFFVQTCREEHKKK HPDASVNFSEFSKKCSERWKTMSAKEKGKFEDMAKADKARYEREMKTYIPPK GET (SEQ ID NO:21), wherein the A box consists of the sequence PTGKMSSYAFF VQTCREEHKKKHPDASVNFSEFSKKCSERWKTMSAKEKGKFEDMAKADKAR YEREMKTYIPPKGET (SEQ ID NO:22)

A polypeptide generated from a GST vector lacking HMGB1 protein was included as a control (containing a GST tag only). To inactive the bacterial DNA that bound to the wild type HMGB1 and some of the mutants (carboxy terminus and B box), DNase I (Life Technologies), for carboxy terminus and B box mutants, or benzonase nuclease (Novagen, Madison, Wis.), for wild type HMGB1, was added at about 20 units/ml bacteria lysate. Degradation of DNA was verified by ethidium bromide staining of the agarose gel containing HMGB1 proteins before and after the treatment. The protein eluates were passed over a polymyxin B column (Pierce, Rockford, Ill.) to remove any contaminating LPS, and dialyzed extensively against phosphate buffered saline to remove excess reduced glutathione. The preparations were then lyophilized and redissolved in sterile water before use. LPS levels were less than 60 pg/μg protein for all of the mutants and 300 pg/μg for wild type HMG-1, as measured by Limulus amebocyte lysate assay (Bio Whittaker Inc., Walkersville, Md.). The integrity of protein was verified by SDS-PAGE. Recombinant rat HMGB1 (Wang et al., Science 285: 248-251, 1999) was used in some experiments since it does not have degraded fragments as observed in purified human HMGB1.

Peptide Synthesis

Peptides were synthesized and HPLC purified at Utah State University Biotechnology Center (Logan, Utah) at 90% purity. Endotoxin was not detectable in the synthetic peptide preparations as measured by Limulus assay.

Cell Culture

Murine macrophage-like RAW 264.7 cells (American Type Culture Collection, Rockville, Md.) were cultured in RPMI 1640 medium (Life Technologies, Grand Island, N.Y.) supplemented with 10% fetal bovine serum (Gemini, Catabasas, Calif.), penicillin and streptomycin (Life Technologies) and were used at 90% confluence in serum-free Opti-MEM I medium (Life Technologies, Grand Island, N.Y.). Polymyxin B (Sigma, St. Louis, Mo.) was routinely added at 100-1,000 units/ml to neutralize the activity of any contaminating LPS as previously described; polymyxin B alone did not influence cell viability assessed with trypan blue (Wang et al., supra). Polymyxin B was not used in experiments of synthetic peptide studies.

Measurement of TNF Release From Cells

TNF release was measured by a standard murine fibroblast L929 (ATCC, American Type Culture Collection, Rockville, Md.) cytotoxicity bioassay (Bianchi et al., Journal of Experimental Medicine 183:927-936, 1996) with the minimum detectable concentration of 30 pg/ml. Recombinant mouse TNF was obtained from R&D system Inc., (Minneapolis, Minn.). Murine fibroblast L929 cells (ATCC) were cultured in DMEM (Life Technologies, Grand Island, N.Y.) supplemented with 10% fetal bovine serum (Gemini, Catabasas, Calif.), penicillin (50 units/ml) and streptomycin (50 μg/ml) (Life Technologies) in a humidified incubator with 5% CO₂.

Antibody Production

Polyclonal antibodies against HMGB1 B box were raised in rabbits (Cocalico Biologicals, Inc., Reamstown, Pa.) and assayed for titer by immunoblotting. IgG was purified from anti-HMGB1 antiserum using Protein A agarose according to manufacturer's instructions (Pierce, Rockford, Ill.). Anti-HMGB1 B box antibodies were affinity purified using cyanogen bromide activated Sepharose beads (Cocalico Biological, Inc.). Non-immune rabbit IgG was purchased from Sigma (St. Louis, Mo.). Antibodies detected full length HMGB1 and B box in immunoassay, but did not cross react with TNF, IL-1 and IL-6.

Labeling of HMGB1 with Na-¹²⁵I and Cell Surface Binding

Purified HMGB1 protein (10 μg) was radiolabeled with 0.2 mCi of carrier-free ¹²⁵I (NEN Life Science Products Inc., Boston, Mass.) using Iodo-beads (Pierce, Rockford, Ill.) according to the manufacturer's instructions. ¹²⁵I-HMGB1 protein was separated from un-reacted ¹²⁵I by gel chromatography columns (P6 Micro Bio-Spin Chromatography Columns, Bio-Rad Laboratories, Hercules, Calif.) previously equilibrated with 300 mM sodium chloride, 17.5 mM sodium citrate, pH 7.0, and 0.1% bovine serum albumin (BSA). The specific activity of the eluted HMGB1 was about 2.8×10⁶ cpm/μg protein. Cell surface binding studies were performed as previously described (Yang et al., Am. J. Physiol. 275:C675-C683, 1998). RAW 264.7 cells were plated on 24-well dishes and grown to confluence. Cells were washed twice with ice-cold PBS containing 0.1% BSA and binding was carried out at 4° C. for 2 hours with 0.5 ml binding buffer containing 120 mM sodium chloride, 1.2 mM magnesium sulfate, 15 mM sodium acetate, 5 mM potassium chloride, 10 mM Tris.HCl, pH 7.4, 0.2% BSA, 5 mM glucose and 25,000 cpm ¹²⁵I-HMGB1. At the end of the incubation the supernatants were discarded and the cells were washed three times with 0.5 ml of ice-cold PBS with 0.1% BSA and lysed with 0.5 ml of 0.5 N NaOH and 0.1% SDS for 20 minutes at room temperature. The radioactivity in the lysate was then measured using a gamma counter. Specific binding was determined as total binding minus the radioactivity obtained in the presence of an excess amount of unlabeled HMGB1 or A box proteins.

Animal Experiments

TNF knock out mice were obtained from Amgen (Thousand Oaks, Calif.) and were on a B6x129 background. Age-matched wild-type B6x129 mice were used as a control for the studies. Mice were bred in-house at the University of Florida specific pathogen-free transgenic mouse facility (Gainesville, Fla.) and were used at 6-8 weeks of age.

Male 6-8 week old Balb/c and C3H/HeJ mice were purchased from Harlen Sprague-Dawley (Indianapolis, Ind.) and were allowed to acclimate for 7 days before use in experiments. All animals were housed in the North Shore University Hospital Animal Facility under standard temperature, and a light and dark cycle.

Cecal Ligation and Puncture

Cecal ligation and puncture (CLP) was performed as described previously (Fink and Heard, J. Surg. Res. 49:186-196, 1990; Wichmann et al., Crit. Care Med. 26:2078-2086, 1998; and Remick et al., Shock 4:89-95, 1995). Briefly, Balb/c mice were anesthetized with 75 mg/kg ketamine (Fort Dodge, Fort Dodge, Iowa) and 20 mg/kg of xylazine (Bohringer Ingelheim, St. Joseph, Mo.) intramuscularly. A midline incision was performed, and the cecum was isolated. A 6-0 prolene suture ligature was placed at a level 5.0 mm from the cecal tip away from the ileocecal valve.

The ligated cecal stump was then punctured once with a 22-gauge needle, without direct extrusion of stool. The cecum was then placed back into its normal intra-abdominal position. The abdomen was then closed with a running suture of 6-0 prolene in two layers, peritoneum and fascia separately to prevent leakage of fluid. All animals were resuscitated with a normal saline solution administered sub-cutaneously at 20 ml/kg of body weight. Each mouse received a subcutaneous injection of imipenem (0.5 mg/mouse) (Primaxin, Merck & Co., Inc., West Point, Pa.) 30 minutes after the surgery. Animals were then allowed to recuperate. Mortality was recorded for up to 1 week after the procedure; survivors were followed for 2 weeks to ensure no late mortalities had occurred.

D-Galactosamine Sensitized Mice

The D-galactosamine-sensitized model has been described previously (Galanos et al., Proc Natl. Acad. Sci. USA 76: 5939-5943, 1979; and Lehmann et al., J. Exp. Med. 165: 657-663, 1997). Mice were injected intraperitoneally with 20 mg D-galactosamine-HCL (Sigma)/mouse (in 200 μl PBS) and 0.1 or 1 mg of either HMGB1 B box or vector protein (in 200 μl PBS). Mortality was recorded daily for up to 72 hours after injection; survivors were followed for 2 weeks, and no later deaths from B box toxicity were observed.

Spleen Bacteria Culture

Fourteen mice received either anti-HMGB1 antibody (n=7) or control (n=7) at 24 and 30 hours after CLP, as described herein, and were euthanized for necropsy. Spleen bacteria were recovered as described previously (Villa et al., J. Endotoxin Res. 4:197-204, 1997). Spleens were removed using sterile technique and homogenized in 2 ml PBS. After serial dilutions with PBS, the homogenate was plated as 0.15 ml aliquots on tryptic soy agar plates (Difco, Detroit, Mich.) and CFU were counted after overnight incubation at 37° C.

Statistical Analysis

Data are presented as mean±SEM unless otherwise stated. Differences between groups were determined by two-tailed Student's t-test, one-way ANOVA followed by the least significant difference test or 2 tailed Fisher's Exact Test.

### EXAMPLE 2

### Mapping the HMGB1 Domains for Promotion of Cytokine Activity

HMGB1 has 2 folded DNA binding domains (A and B boxes) and a negatively charged acidic carboxyl tail). To elucidate the structural basis of HMGB1 cytokine activity, and to map the inflammatory protein domain, we expressed full length and truncated forms of HMGB1 by mutagenesis and screened the purified proteins for stimulating activity in monocyte cultures (FIG. 1). Full length HMGB1, a mutant in which the carboxy terminus was deleted, a mutant containing only the B box, and a mutant containing only the A box were generated. These mutants of human HMGB1 were made by polymerase chain reaction (PCR) using specific primers as described herein, and the mutant proteins were expressed using a glutathione S-transferase (GST) gene fusion system (Pharmacia Biotech, Piscataway, N.J.) in accordance with the manufacturer's instructions. Briefly, DNA fragments, made by PCR methods, were fused to GST fusion vectors and amplified in E. Coli. The expressed HMGB1 protein and HMGB1 mutants were then isolated using a GST affinity column.

The effect of the mutants on TNF release from Murine macrophage-like RAW 264.7 cells (ATCC) was carried out as follows. RAW 264.7 cells were cultured in RPMI 1640 medium (Life Technologies, Grand Island, N.Y.) supplemented with 10% fetal bovine serum (Gemini, Catabasas, Calif.), penicillin and streptomycin (Life Technologies). Polymyxin (Sigma, St. Louis, Mo.) was added at 100 units/ml to suppress the activity of any contaminating LPS. Cells were incubated with 1 μg/ml of full length (wild-type) HMGB1 and each HMGB1 mutant protein in Opti-MEM I medium for 8 hours. Conditioned supernatants (containing TNF which had been released from the cells) were collected and TNF released from the cells was measured by a standard murine fibroblast L929 (ATCC) cytotoxicity bioassay (Bianchi et al., supra) with the minimum detectable concentration of 30 pg/ml. Recombinant mouse TNF was obtained from R & D Systems Inc., (Minneapolis, Minn.) and used as control in these experiments. The results of this study are shown in FIG. 1. Data in FIG. 1 are all presented as mean±SEM unless otherwise indicated. (N=6−10).

As shown in FIG. 1, wild-type HMGB1 and carboxyl-truncated HMGB1 significantly stimulated TNF release by monocyte cultures (murine macrophage-like RAW 264.7 cells). The B box was a potent activator of monocyte TNF release. This stimulating effect of the B box was specific, because A box only weakly activated TNF release.

### EXAMPLE 3

### HMGB1 B Box Protein Promotes Cytokine Activity in a Dose Dependent Manner

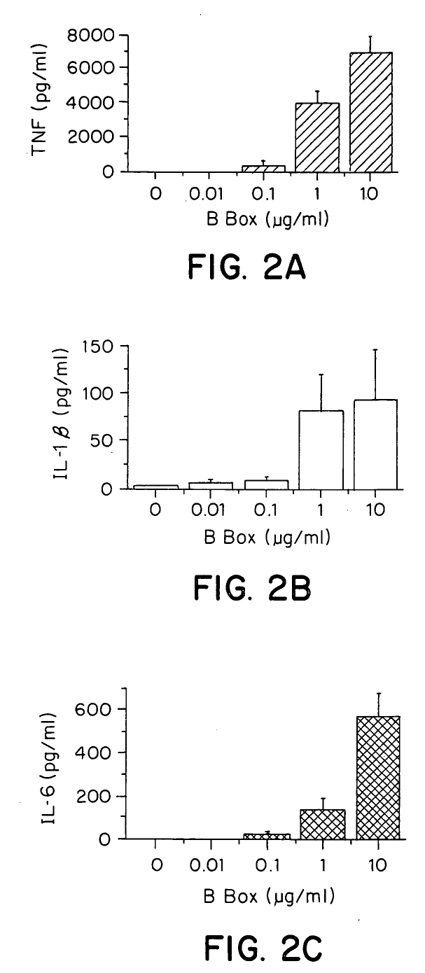

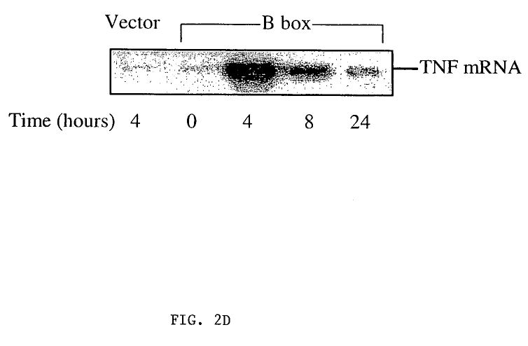

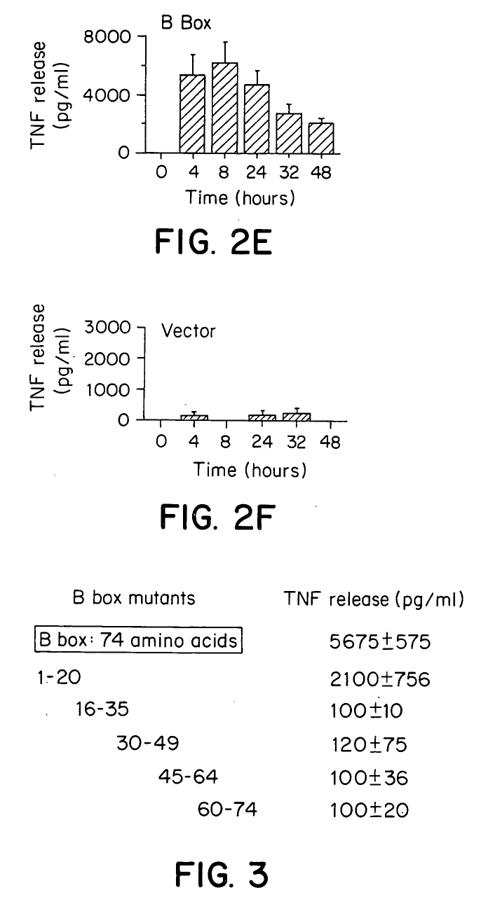

To further examine the effect of HMGB1 B box on cytokine production, varying amounts of HMGB1 B box were evaluated for the effects on TNF, IL-1B, and IL-6 production in murine macrophage-like RAW 264.7 cells. RAW 264.7 cells were stimulated with HMGB B box protein at 0-10 μg/ml, as indicated in FIGS. 2A-2C for 8 hours. Conditioned media were harvested and measured for TNF, IL-1β and IL-6 levels. TNF levels were measured as described herein, and IL-1β and IL-6 levels were measured using the mouse IL-1β and IL-6 enzyme-linked immunosorbent assay (ELISA) kits (R&D System Inc., Minneapolis, Minn.) and N>5 for all experiments. The results of the studies are shown in FIGS. 2A-2C.

As shown in FIG. 2A, TNF release from RAW 264.7 cells increased with increased amounts of B box administered to the cells. As shown in FIG. 2B, addition of 1 μg/ml or 10 μg/ml of B box resulted in increased release of IL-1β from RAW 264.7 cells. In addition, as shown in FIG. 2C, IL-6 release from RAW 264.7 cells increased with increased amounts of B box administered to the cells.

The kinetics of B box-induced TNF release were also examined. TNF release and TNF mRNA expression were measured in RAW 264.7 cells induced by B box polypeptide or GST tag polypeptide only (used as a control (vector)) (10 μg/ml) for 0 to 48 hours. Supernatants were analyzed for TNF protein levels by an L929 cytotoxicity assay (N=3-5) as described herein. For mRNA measurement, cells were plated in 100 mm plates and treated in Opti-MEM I medium containing B box polypeptide or the vector alone for 0, 4, 8, or 24 hours, as indicated in FIG. 2D. The vector only sample was assayed at the 4 hour time point. Cells were scraped off the plate and total RNA was isolated using the RNAzol B method in accordance with the manufacturer's instructions (Tel-Test “B”, Inc., Friendswood, Tex.). TNF (287 bp) was measured by RNase protection assay (Ambion, Austin, Tex.). Equal loading and the integrity of RNA was verified by ethidium bromide staining of the RNA sample on an agarose-formaldehyde gel. The results of the RNase protection assay are shown in FIG. 2D. As shown in FIG. 2D, B box activation of monocytes occurred at the level of gene transcription, because TNF mRNA was increased significantly in monocytes exposed to B box protein (FIG. 2B). TNF mRNA expression was maximal at 4 hours and decreased at 8 and 24 hours. The vector only control (GST tag) showed no effect on TNF mRNA expression. A similar study was carried out measuring TNF protein released from RAW 264.7 cells 0, 4, 8, 24, 32 or 48 hours after administration of B box or vector only (GST tag), using the L929 cytotoxicity assay described herein. Compared to the control (medium only), B box treatment stimulated TNF protein expression (FIG. 2E) and vector alone (FIG. 2F) did not. Data are representative of three separate experiments. Together these data indicate that the HMGB1 B box domain has cytokine activity and is responsible for the cytokine stimulating activity of full length HMGB1.

In summary, the HMGB1 B box dose-dependently stimulated release of TNF, IL-1β and IL-6 from monocyte cultures (FIGS. 2A-2C), in agreement with the inflammatory activity of full length HMGB1 (Andersson et al., J. Exp. Med. 192: 565-570, 2000). In addition, these studies indicate that maximum TNF protein release occurred within 8 hours (FIG. 2E). This delayed pattern of TNF release is similar to TNF release induced by HMGB1 itself, and is significantly later than the kinetics of TNF induced by LPS (Andersson et al., supra).

### EXAMPLE 4

### The First 20 Amino Acids of the HMGB1 B Box Stimulate TNF Activity

The TNF-stimulating activity of the HMGB1 B box was further mapped. This study was carried out as follows. Fragments of the B box were generated using synthetic peptide protection techniques, as described herein. Five HMGB1 B box fragments (from SEQ ID NO:20), containing amino acids 1-20, 16-25, 30-49, 45-64, or 60-74 of the HMGB1 B box were generated, as indicated in FIG. 3. RAW 264.7 cells were treated with B box (1 μg/ml) or a synthetic peptide fragment of the B box (10 μg/ml), as indicated in FIG. 3, for 10 hours and TNF release in the supernatants was measured as described herein. Data shown are mean±SEM, (n=3 experiments, each done in duplicate and validated using 3 separate lots of synthetic peptides). As shown in FIG. 3, TNF-stimulating activity was retained by a synthetic peptide corresponding to amino acids 1-20 of the HMGB1 B box of SEQ ID NO:20 (fkdpnapkrlpsafflfcse; SEQ ID NO:23). The TNF stimulating activity of the 1-20-mer was less potent than either the full length synthetic B box (1-74-mer), or full length HMGB1, but the stimulatory effects were specific because the synthetic 20-mers for amino acid fragments containing 16-25, 30-49, 45-64, or 60-74 of the HMGB1 B box did not induce TNF release. These results are direct evidence that the macrophage stimulating activity of the B box specifically maps to the first 20 amino acids of the HMGB B box domain of SEQ ID NO:20). This B box fragment can be used in the same manner as a polypeptide encoding a full length B box polypeptide, for example, to stimulate release of a proinflammatory cytokine, or to treat a condition in a patient characterized by activation of an inflammatory cytokine cascade.

### EXAMPLE 5

### HMGB1 A Box Protein Antagonizes HMGB1 Induced Cytokine Activity in a Dose Dependent Manner

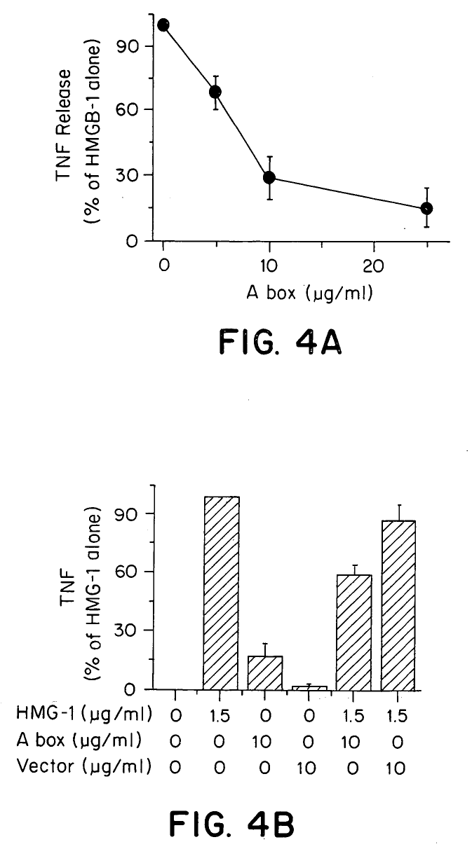

Weak agonists are by definition antagonists. Since the HMGB1 A box only weakly induced TNF production, as shown in FIG. 1, the ability of HMGB1 A box to act as an antagonist of HMGB1 activity was evaluated. This study was carried out as follows. Sub-confluent RAW 264.7 cells in 24-well dishes were treated with HMGB1 (1 μg/ml) and 0, 5, 10, or 25 μg/ml of A box for 16 hours in Opti-MEM I medium in the presence of polymyxin B (100 units/ml). The TNF-stimulating activity (assayed using the L929 cytotoxicity assay described herein) in the sample receiving no A box was expressed as 100%, and the inhibition by A box was expressed as percent of HMGB1 alone. The results of the effect of A box on TNF release from RAW 264.7 cells is shown in FIG. 4A. As shown in FIG. 4A, the A box dose-dependently inhibited HMGB1 induced TNF release with an apparent EC₅₀ of approximately 7.5 μg/ml. Data in FIG. 4A are presented as mean±SD (n=2-3 independent experiments).

### EXAMPLE 6

### HMGB1 A Box Protein Inhibits Full Length HMGB1 and HMGB1 B Box Cytokine Activity

Antagonism of full length HMGB1 activity by HMGB1 A box or GST tag (vector control) was also determined by measuring TNF release from RAW 264.7 macrophage cultures stimulated by co-addition of A box with full length HMGB1. RAW 264.7 macrophage cells (ATCC) were seeded into 24-well tissue culture plates and used at 90% confluence. The cells were treated with HMGB1, and/or A boxes as indicated for 16 hours in Optimum I medium (Life Technologies, Grand Island, N.Y.) in the presence of polymyxin B (100 units/ml, Sigma, St. Louis, Mo.) and supernatants were collected for TNF measurement (mouse ELISA kit from R&D System Inc, Minneapolis, Minn.). TNF-inducing activity was expressed as a percentage of the activity achieved with HMGB1 alone. The results of these studies are shown in FIG. 4B. FIG. 4B is a histogram of the effect of HMGB1 (HMG-1), alone, A box alone, Vector (control) alone, HMGB1 in combination with A box, and HMGB1 in combination with vector. As shown in FIG. 4B, HMGB1 A box significantly attenuated the TNF stimulating activity of full length HMGB1.

### EXAMPLE 7

### HMGB1 A Box Protein Inhibits HMGB1 Cytokine Activity by Binding to It

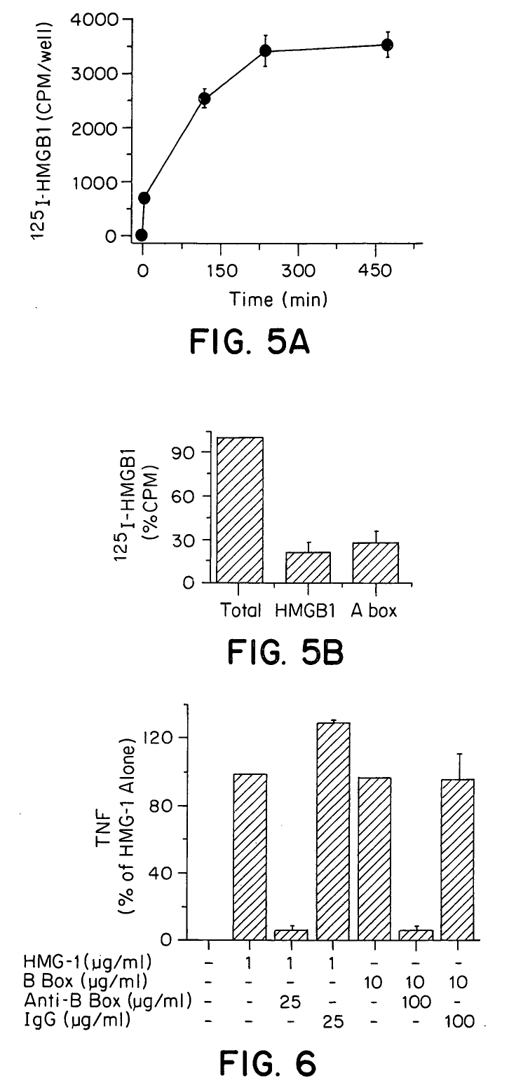

To determine whether the HMGB1 A box acts as an antagonist by displacing HMGB1 binding, ¹²⁵I-labeled —HMGB1 was added to macrophage cultures and binding was measured at 4° C. after 2 hours. Binding assays in RAW 264.7 cells were performed as described herein. ¹²⁵I-HMGB1 binding was measured in RAW 264.7 cells plated in 24-well dishes for the times indicated in FIG. 5A. Specific binding shown equals total cell-associated ¹²⁵I-HMGB1 (CPM/well) minus cell associated CPM/well in the presence of 5,000 fold molar excess of unlabeled HMGB1. FIG. 5A is a graph of the binding of ¹²⁵I-HMGB1 over time. As shown in FIG. 5A, HMGB1 exhibited saturable first order binding kinetics. The specificity of binding was assessed as described in Example 1.

In addition, ¹²⁵I-HMG-1 binding was measured in RAW 264.7 cells plated on 24-well dishes and incubated with ¹²⁵I HMGB1 alone or in the presence of unlabeled HMGB1 or A box. The results of this binding assay are shown in FIG. 5B. Data represents mean±SEM from 3 separate experiments. FIG. 5B is a histogram of the cell surface binding of ¹²⁵1-HMGB1 in the absence of unlabeled HMGB1 or HMGB1 A box, or in the presence of 5,000 molar excess of unlabeled HMGB1 or HMGB1 A box, measured as a percent of the total CPM/well. In FIG. 5B, “Total” equals counts per minutes (CPM)/well of cell associated ¹²⁵I-HMGB1 in the absence of unlabeled HMGB1 or A box for 2 hours at 4° C. “HMGB1” or “A box” equals CPM/well of cell-associated ¹²⁵I-HMGB1 in the presence of 5,000 molar excess of unlabeled HMGB1 or unlabeled A box. The data are expressed as the percent of total counts obtained in the absence of unlabeled HMGB1 proteins (2,382,179 CPM/well). These results indicate that the HMGB1 A box is a competitive antagonist of HMGB1 activity in vitro and inhibits the TNF-stimulating activity of HMGB1.

### EXAMPLE 8

### Inhibition of Full Length HMGB1 and HMGB1 B Box Cytokine Activity by Anti-B Box Polyclonal Antibodies.

The ability of antibodies directed against the HMGB1 B box to modulated the effect of full length or HMGB1 B box was also assessed. Affinity purified antibodies directed against the HMGB1 B box (B box antibodies) were generated as described herein and using standard techniques. To assay the effect of the antibodies on HMGB1-induced or HMGB1 B box-induced TNF release from RAW 264.7 cells, sub-confluent RAW 264.7 cells in 24-well dishes were treated with HMGB1 (HMG-1; 1 μg/ml) or HMGB1 B box (B Box; 10 μg/ml) for 10 hours with or without anti-B box antibody (25 μg/ml or 100 μg/ml antigen affinity purified, Cocalico Biologicals, Inc., Reamstown, Pa.) or non-immune IgG (25 μg/ml or 100 μg/ml; Sigrna) added. TNF release from the RAW 264.7 cells was measured using the L929 cytotoxicity assay as described herein. The results of this study are shown in FIG. 6, which is a histogram of TNF released by RAW 264.7 cells administered nothing, 1 μg/ml of HMGB1, 1 μg/ml of HMGB1 plus 25 μg/ml of anti-B box antibody, 1 μg/ml of HMGB1 plus 25 μg/ml of IgG (control), 10 μg/ml of B-box, 10 μg/ml of B-box plus 100 μg/ml of anti-B box antibody or 10 μg/ml of B-box plus 100 μg/ml of IgG (control). The amount of TNF released from the cells induced by HMGB1 alone (without addition of B box antibodies) was set as 100%, and the data shown in FIG. 6 are the results of 3 independent experiments. As shown in FIG. 6, affinity purified antibodies directed against the HMGB1 B box significantly inhibited TNF release induced by either full length HMGB1 or the HMGB1 B box. These results indicate that such an antibody can be used to modulate HMGB1 function.

### EXAMPLE 9

### HMGB1 B Box Protein is Toxic to D-galactosamine-sensitized Balb/c Mice

To investigate whether the HMGB1 B box has cytokine activity in vivo, we administered HMGB1 B box protein to unanesthetized Balb/c mice sensitized with D-galactosamine (D-gal), a model that is widely used to study cytokine toxicity (Galanos et al., supra). Briefly, mice (20-25 grams, male, Harlan Sprague-Dawley, Indianapolis, Ind.) were intraperitoneally injected with D-gal (20 mg) (Sigma, St. Louis, Mo.) and B box (0.1 mg/ml/mouse or 1 mg/ml/mouse) or GST tag (vector; 0.1 mg/ml/mouse or 1 mg/ml/mouse), as indicated in Table 1. Survival of the mice was monitored up to 7 days to ensure no late death occurred. The results of this study are shown in Table 1.

TABLE 1
Toxicity of HMGB1 B box on D-galactosamine-sensitized
Balb/c Mice

| | Treatment | Alive/total |
|---|---|---|
| Control | — | 10/10 |
| Vector | 0.1 mg/mouse | 2/2 |
| | 1 mg/mouse | 3/3 |
| B box | 0.1 mg/mouse | 6/6 |
| | 1 mg/mouse | 2/8* |

*P < 0.01 versus vector alone as tested by Fisher's Exact Test

The results of this study showed that the HMGB1 B box was lethal to D-galactosamine-sensitized mice in a dose-dependent manner. In all instances in which death occurred, it occurred within 12 hours. Lethality was not observed in mice treated with comparable preparations of the purified GST vector protein devoid of B box.

### EXAMPLE 10

### Histology of D-Galactosamine-Sensitized Balb/c Mice or C3H/HeJ Mice Administered HMGB1 B Box Protein

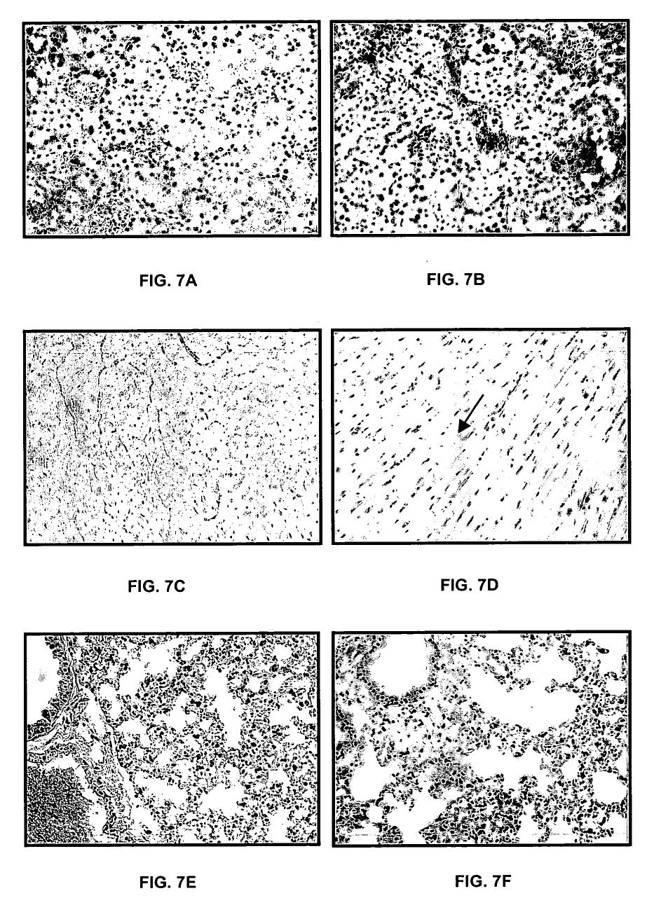

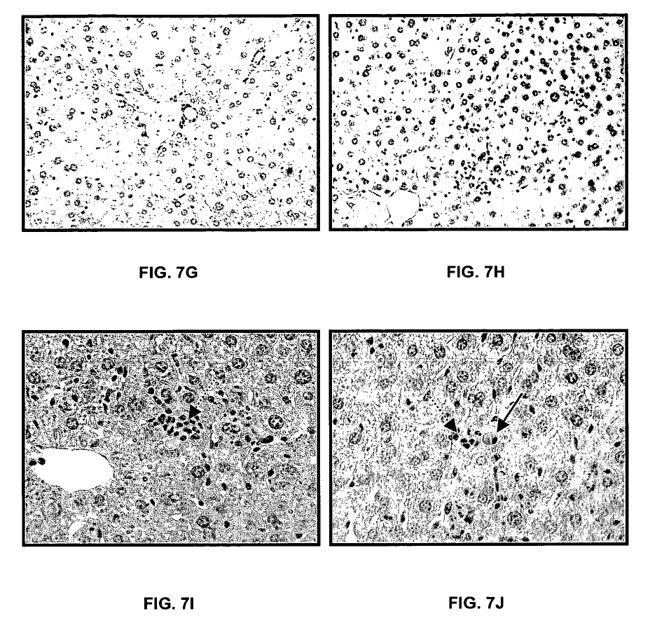

To further assess the lethality of the HMGB1 B box protein in vivo the HMGB1 B box was again administered to D-galactosamine-sensitized Balb/c mice. Mice (3 per group) received D-gal (20 mg/mouse) plus B box or vector (1 mg/mouse) intraperitoneally for 7 hours and were then sacrificed by decapitation. Blood was collected, and organs (liver, heart, kidney and lung) were harvested and fixed in 10% formaldehyde. Tissue sections were prepared with hematoxylin and eosin staining for histological evaluation (Criterion Inc., Vancouver, Canada). The results of these studies are shown in FIGS. 7A-7J, which are scanned images of hematoxylin and eosin stained kidney sections (FIG. 7A), myocardium sections (FIG. 7C), lung sections (FIG. 7E), and liver sections (FIGS. 7G and 7I) obtained from an untreated mouse and kidney sections (FIG. 7B), myocardium sections (FIG. 7D), lung sections (FIG. 7F), and liver sections (FIGS. 7H and 7J) obtained from mice treated with the HMGB1 B box. Compared to the control mice, B box treatment caused no abnormality in kidneys (FIGS. 7A and 7B) and lungs (FIGS. 7E and 7F). The mice had some ischemic changes and loss of cross striation in myocardial fibers in the heart (FIGS. 7C and 7D as indicated by the arrow in FIG. 7D). Liver showed most of the damage by the B box as illustrated by active hepatitis (FIGS. 7G-7J). In FIG. 7J, hepatocyte dropouts are seen surrounded by accumulated polymorphonuclear leukocytes. The arrows in FIG. 7J point to the sites of polymorphonuclear accumulation (dotted) or apoptotic hepatocytes (solid). Administration of HMGB1 B box in vivo also stimulated significantly increased serum levels of IL-6 (315+93 vs.20+7 pg/ml, B box vs. control, p<0.05) and IL-1β (15+3 vs. 4+1 pg/ml, B box vs. control, p<0.05).

Administration of B box protein to C3H/HeJ mice (which do not respond to endotoxin) was also lethal, indicating that HMGB1 B box is lethal in the absence of LPS signal transduction. Hematoxylin and eosin stained sections of lung and kidney collected 8 hours after administration of B box revealed no abnormal morphologic changes. Examination of sections from the heart however, revealed evidence of ischemia with loss of cross striation associated with amorphous pink cytoplasm in myocardial fibers. Sections from liver showed mild acute inflammatory responses, with some hepatocyte dropout and apoptosis, and occasional polymorphonuclear leukocytes. These specific pathological changes were comparable to those observed after administration of full length HMGB1 and confirm that the B box alone can recapitulate the lethal pathological response to HMGB1 in vivo.

To address whether the TNF-stimulating activity of HMGB1 contributes to the mediation of lethality by B box, we measured lethality in TNF knock-out mice (TNF-KO, Nowak et al., Am. J. Physiol. Regul. Integr. Comp. Physiol. 278: R1202-R1209, 2000) and the wild-type controls (B6×129 strain) sensitized with D-galactosamine (20 mg/mouse) and exposed to B box (1 mg/mouse, injected intraperitoneally). The B box was highly lethal to the wild-type mice (6 dead out of nine exposed) but lethality was not observed in the TNF-KO mice treated with B box (0 dead out of 9 exposed, p<0.05 v. wild type). Together with the data from the RAW 264.7 macrophage cultures, described herein, these data now indicate that the B box of HMGB1 confers specific TNF-stimulating cytokine activity.

### EXAMPLE 11

### HMGB1 Protein Level is Increased in Septic Mice

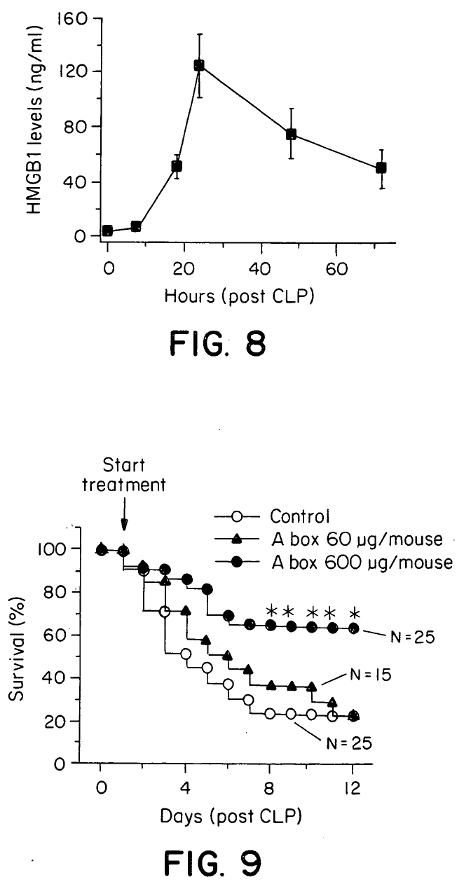

To examine the role of HMGB1 in sepsis, we established sepsis in mice and measured serum HMGB1 using a quantitative immunoassay described previously (Wang et al., supra). Mice were subjected to cecal ligation and puncture (CLP), a well characterized model of sepsis caused by perforating a surgically-created cecal diverticulum, that leads to polymicrobial peritonitis and sepsis (Fink and Heard, supra; Wichmann et al., supra; and Remick et al., supra). Serum levels of HMGB1 were then measured (Wang et al., supra). FIG. 8 shows the results of this study in a graph that illustrates the levels of HMGB1 in mice 0 hours, 8 hours, 18 hours, 24 hours, 48 hours, and 72 hours after subjection to CLP. As shown in FIG. 8, serum HMGB1 levels were not significantly increased for the first eight hours after cecal perforation, then increased significantly after 18 hours (FIG. 8). Increased serum HMGB1 remained at elevated plateau levels for at least 72 hours after CLP, a kinetic profile that is quite similar to the previously-described, delayed HMGB1 kinetics in endotoxemia (Wang et al., supra). This temporal pattern of HMGB1 release corresponded closely to the development of signs of sepsis in the mice. During the first eight hours after cecal perforation the animals were observed to be mildly ill, with some diminished activity and loss of exploratory behavior. Over the ensuing 18 hours the animals became gravely ill, huddled together in groups with piloerection, did not seek water or food, and became minimally responsive to external stimuli or being examined by the handler.

### EXAMPLE 12

### Treatment of Septic Mice with HMGB1 A Box Protein Increases Survival of Mice

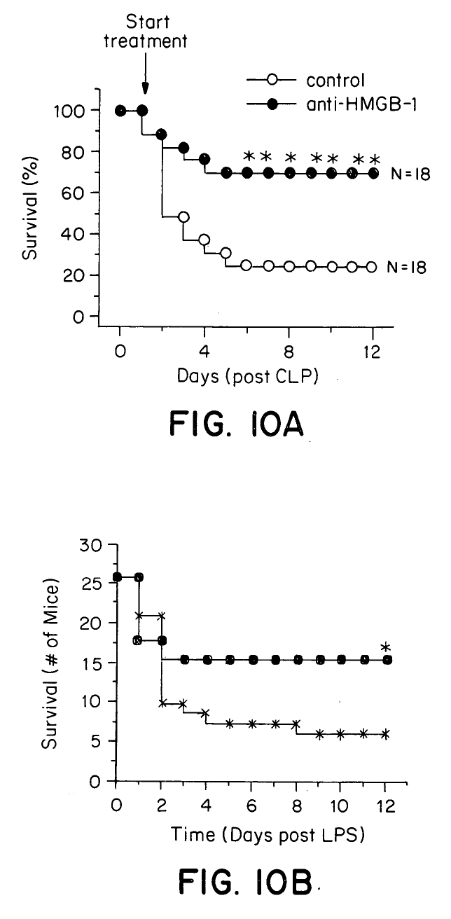

To determine whether the HMGB1 A box can inhibit the lethality of HMGB1 during sepsis, mice were subjected to cecal perforation and treated by administration of A box beginning 24 hours after the onset of sepsis. CLP was performed on male Balb/c mice as described herein. Animals were randomly grouped, with 15-25 mice per group. The HMGB1 A box (60 or 600 μg/mouse each time) or vector (GST tag, 600 μg/mouse) alone was administered intraperitoneally twice daily for 3 days beginning 24 hours after CLP. Survival was monitored twice daily for up to 2 weeks to ensure no late death occurred. The results of this study are illustrated in FIG. 9, which is a graph of the effect of vector (GST; control) 60 μg/mouse or 600 μg/mouse on survival over time (*P<0.03 vs. control as tested by Fisher's exact test). As shown in FIG. 9, administration of the HMGB1 A box significantly rescued mice from the lethal effects of sepsis, and improved survival from 28% in the animals treated with protein purified from the vector protein (GST) devoid of the A box, to 68% in animals receiving A box (P<0.03 by Fischer's exact test). The rescuing effects of the HMGB1 A box in this sepsis model were A box dose-dependent; animals treated with 600 μg/mouse of A box were observed to be significantly more alert, active, and to resume feeding behavior as compared to either control animals treated with vector-derived preparations, or to animals treated with only 60 μg of A box. The latter animals remained gravely ill, with depressed activity and feeding for several days, and most died.

### EXAMPLE 13

### Treatment of Septic Mice with Anti-HMGB1 Antibody Increases Survival of Mice

Passive immunization of critically ill septic mice with anti-HMGB1 antibodies was also assessed. In this study, male Balb/c mice (20-25 μm) were subjected to CLP, as described herein. Affinity purified anti-HMGB1 B box polyclonal antibody or rabbit IgG (as control) was administered at 600 μg/mouse beginning 24 hours after the surgery, and twice daily for 3 days. Survival was monitored for 2 weeks. The results of this study are shown in FIG. 10A, which is a graph of the survival of septic mice treated with either a control antibody or an anti-HMGB1 antibody. The results show that anti-HMGB1 antibodies administered to the mice 24 hours after the onset of cecal perforation significantly rescued animals from death as compared to administration of non-immune antibodies (p<0.02 by Fisher's exact test). Within 12 hours after administration of anti-HMGB1 antibodies, treated animals showed increased activity and responsiveness as compared to controls receiving non-immune antibodies. Whereas animals treated with non-immune antibodies remained huddled, ill kempt, and inactive, the treated animals improved significantly and within 48 hours resumed normal feeding behavior. Anti-HMGB1 antibodies did not suppress bacterial proliferation in this model, because we observed comparable bacterial counts (CFU, the aerobic colony forming units) from spleens 31 hours after CLP in the treated animals as compared to animals receiving irrelevant antibodies (control bacteria counts=3.5+0.9×10⁴ CFU/g; n=7). Animals were monitored for up to 2 weeks afterwards, and late deaths were not observed, indicating that treatment with anti-HMGB1 conferred complete rescue from lethal sepsis, and did not merely delay death.

To our knowledge, no other specific cytokine-directed therapeutic is as effective when administered so late after the onset of sepsis. By comparison, administration of anti-TNF actually increases mortality in this model, and anti-MIF antibodies are ineffective if administered more than 8 hours after cecal perforation (Remick et al, supra; and Calandra et al., Nature Med. 6:164-170, 2000). These data demonstrate that HMGB1 can be targeted as late as 24 hours after cecal perforation in order to rescue lethal cases of established sepsis.

In another example of the rescue of endotoxemic mice using anti-B box antibodies, anti-HMGB1 B box antibodies were evaluated for their ability to rescue LPS-induced septic mice. Male Balb/c mice (20-25 μm, 26 per group) were treated with an LD75 dose of LPS (15 mg/kg) injected intraperitoneally (IP). Anti-HMGB1 B box or non-immune rabbit serum (0.3 ml per mouse each time, IP) was given at time 0, +12 hours and +24 hours after LPS administration. Survival of mice was evaluated over time. The results of this study are shown in FIG. 10B, which is a graph of the survival of septic mice administered anti-HMGB1 B box antibodies or non-immune serum. As shown in FIG. 10B, anti-HMGB1 B box antibodies improved survival of the septic mice.

### EXAMPLE 14

### Inhibition of HMGB1 Signaling Pathway Using an Anti-RAGE Antibody

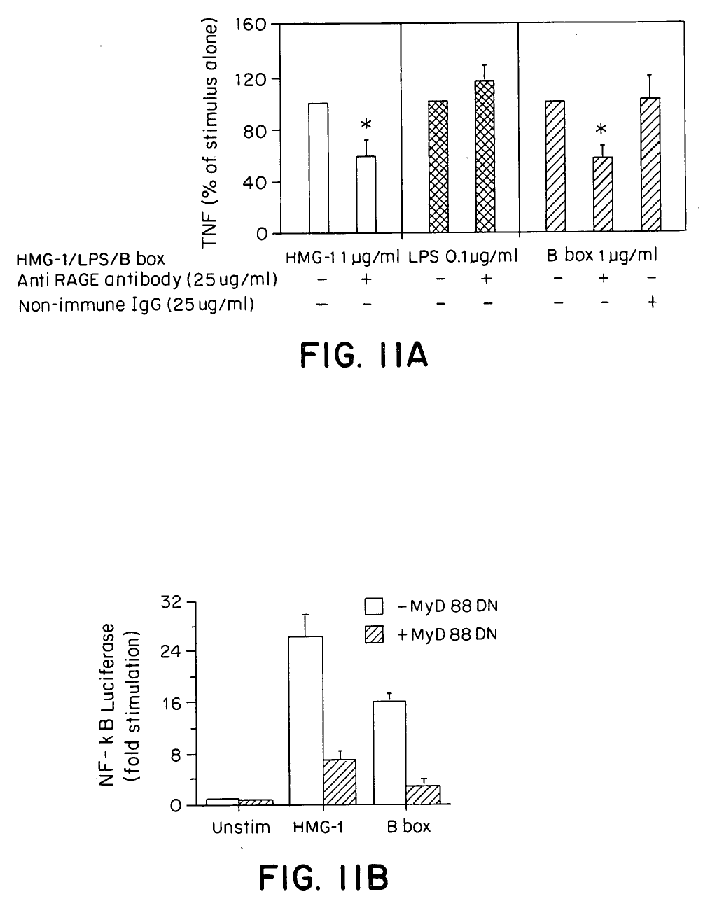

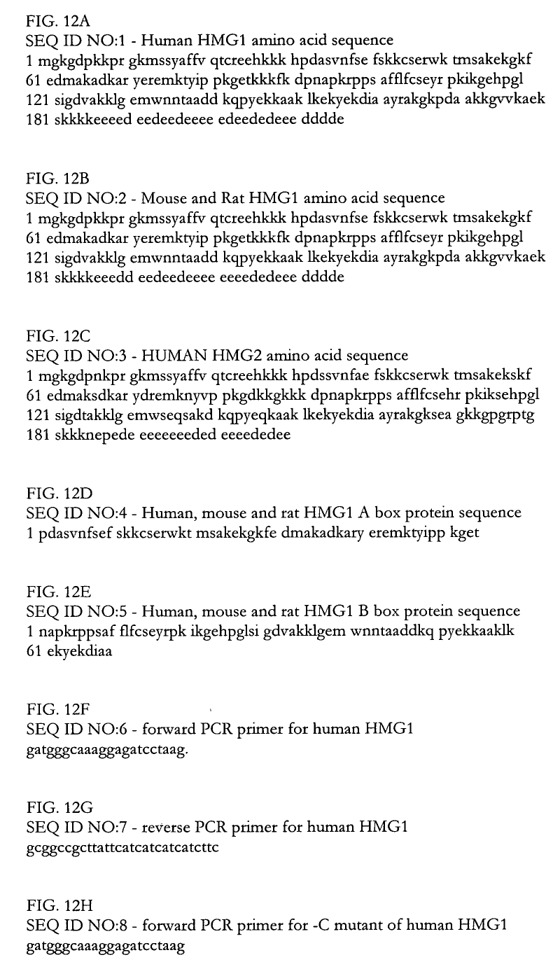

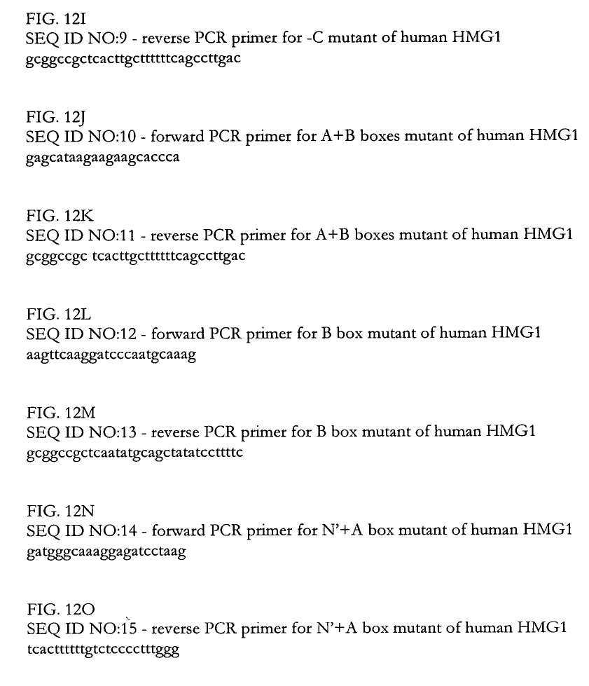

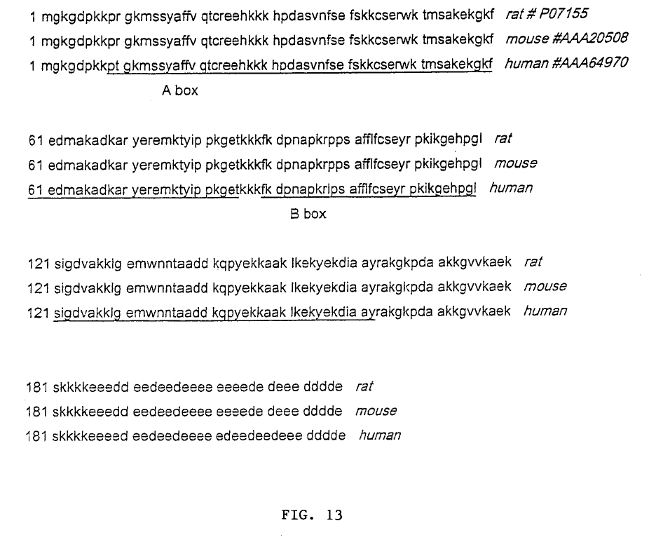

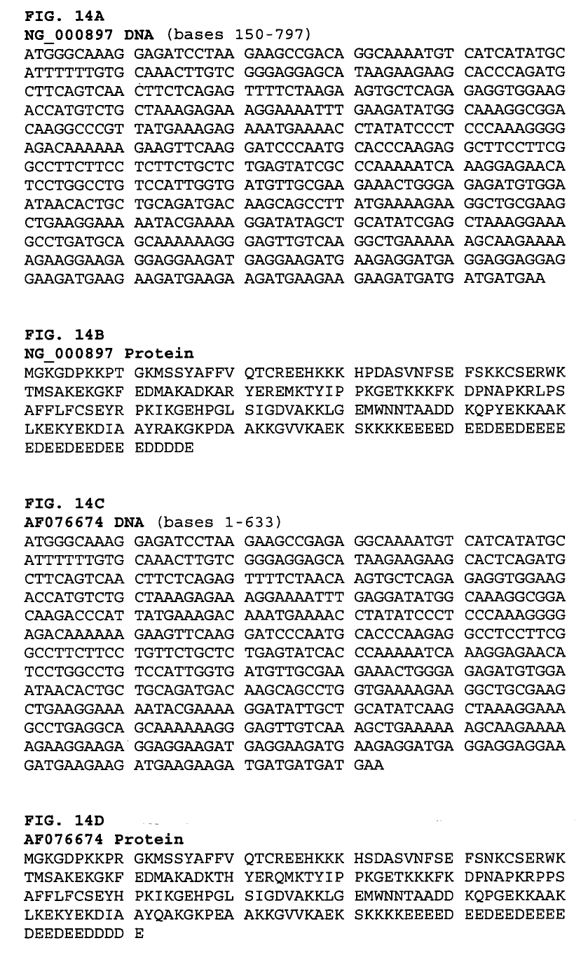

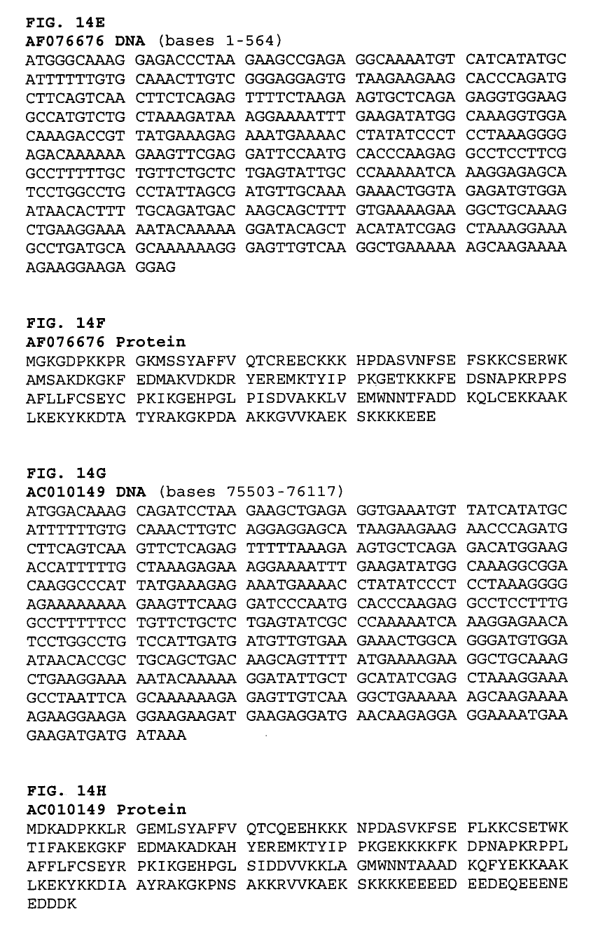

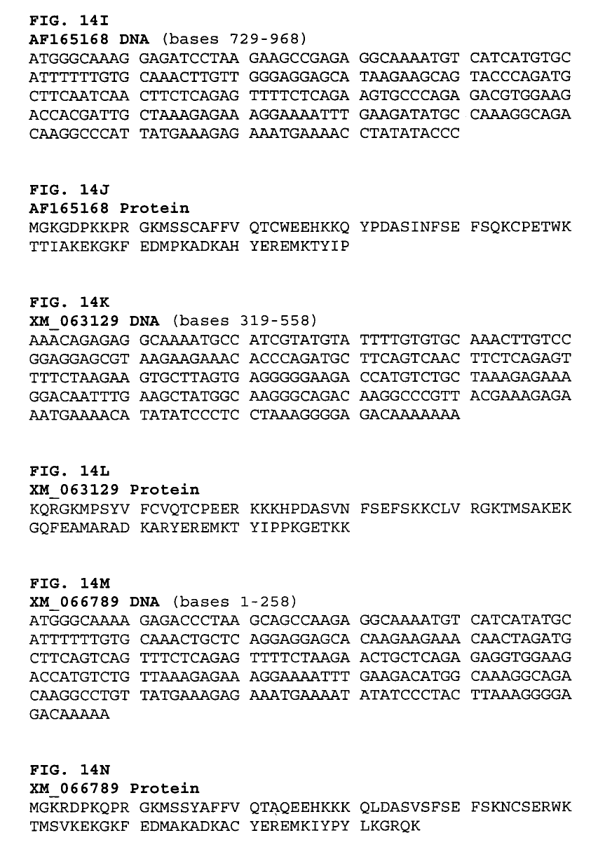

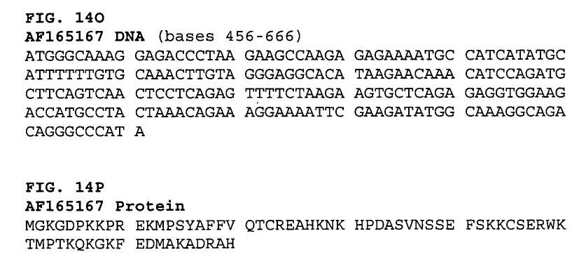

Previous data implicated RAGE as an HMGB1 receptor that can mediate neurite outgrowth during brain development and migration of smooth muscle cells in wound healing (Hori et al. J. Biol. Chem. 270:25752-25761, 1995; Merenmies et al. J. Biol. Chem. 266:16722-16729, 1991; and Degryse et al., J. Cell Biol. 152:1197-1206, 2001). We measured TNF release in RAW 264.7 cultures stimulated with HMGB1 (1 μg/ml), LPS (0.1 μg/ml), or HMGB1 B box (1 μg/ml) in the presence of anti-RAGE antibody (25 μg/ml) or non-immune IgG (25 μg/ml). Briefly, the cells were seeded into 24-well tissue culture plates and used at 90% confluence. LPS (E. coli 0111:B4, Sigma, St. Louis, Mo.) was sonicated for 20 minutes before use. Cells were treated with HMGB1 (HMG-1; 1 μg/ml), LPS (0.1 μg/ml), or HMGB1 B box (B Box; 1 μg/ml) in the presence of anti-RAGE antibody (25 μg/ml) or non-immune IgG (25 μg/ml), as indicated in FIG. 11A for 16 hours in serum-free Opti-MEM I medium (Life Technologies) and supernatants were collected for TNF measurement using the L929 cytotoxicity assay described herein. IgG purified polyclonal anti-RAGE antibody (Catalog No. sc-8230, N-16, Santa Cruz Biotech, Inc., Santa Cruz, Calif.) was dialyzed extensively against PBS before use. The results of this study are shown in FIG. 11A, which is a histogram of the effects of HMGB1, LPS, or HMGB1 B box in the presence of anti-RAGE antibodies or non-immune IgG (control) on TNF release from RAW 264.7 cells. As shown in FIG. 11A, compared to non-immune IgG, anti-RAGE antibody significantly inhibited HMGB1 B box-induced TNF release. This suppression was specific, because anti-RAGE did not significantly inhibit LPS-stimulated TNF release. Notably, the maximum inhibitory effect of anti-RAGE decreased HMG-1 signaling by only 40%, suggesting that other signal transduction pathways may participate in HMGB1 signaling.

To examine the effects of HMGB1 or HMGB1 B Box on the NF-κB-dependent ELAM promoter, the following experiment was carried out. RAW 264.7 macrophages were transiently co-transfected with an expression plasmid encoding a murine MyD 88-dominant-negative (DN) mutant (corresponding to amino acids 146-296), or empty vector, plus a luciferase reporter plasmid under the control of the NF-κB-dependent ELAM promoter, as described by Means et al. (J. Immunol. 166:4074-4082, 2001). A portion of the cells were then stimulated with full-length HMGB1 (100 ng/ml), or purified HMGB1 B box (10 μg/ml), for 5 hours. Cells were then harvested and luciferase activity was measured, using standard methods. All transfections were performed in triplicate, repeated at least three times, and a single representative experiment is shown in FIG. 11B. As shown in FIG. 11B, HMGB1 stimulated luciferase activity in samples that were not co-transfected with the MyD 88 dominant negative, and the level of stimulation was decreased in samples that were co-transfected with the MyD 88 dominant negative. This effect was also observed in samples administered HMGB B box.

While this invention has been particularly shown and described with references to preferred embodiments thereof, it will be understood by those skilled in the art that various changes in form and details may be made therein without departing from the scope of the invention encompassed by the appended claims.

What is claimed is:

1. A pharmaceutical composition comprising a polypeptide comprising a high mobility group box (HMGB) A box or a fragment or variant thereof that can inhibit release of a proinflammatory cytokine from a cell treated with high mobility group box (HMGB) protein and an agent that inhibits TNF biological activity, said agent selected from the group consisting of infliximab, etanercept, adalimumab, CDP870, CDP571, Lenercept, and Thalidomide, in a pharmaceutically acceptable carrier.

2. The pharmaceutical composition of claim 1, wherein said polypeptide is a mammalian HMGB A box.

3. The pharmaceutical composition of claim 2, wherein said polypeptide is a mammalian HMGB1 A box.

4. The pharmaceutical composition of claim 3, wherein said polypeptide comprises SEQ ID NO:4.

5. The pharmaceutical composition of claim 4, wherein said polypeptide consists of SEQ ID NO:4.

6. A pharmaceutical composition comprising an antibody that binds an HMGB polypeptide or a biologically active fragment thereof and an agent that inhibits TNF biological activity, said agent selected from the group consisting of infliximab, etanercept, adalimumab, CDP870, CDP571, Lenercept, and Thalidomide, in a pharmaceutically acceptable carrier.

7. The pharmaceutical composition of claim 6, wherein said HMGB polypeptide is a mammalian HMGB polypeptide.

8. The pharmaceutical composition of claim 7, wherein said HMGB polypeptide is an HMGB1 polypeptide.

9. The pharmaceutical composition of claim 8, wherein said HMGB1 polypeptide comprises SEQ ID NO:1.

10. The pharmaceutical composition of claim 9, wherein said HMGB1 polypeptide consists of SEQ ID NO:1.

11. The pharmaceutical composition of claim 6, wherein said biologically active HMGB fragment is an HMGB B box or a biologically active fragment thereof.

12. The pharmaceutical composition of claim 11, wherein said HMGB B box consists of SEQ ID NO:5.

13. The pharmaceutical composition of claim 11, wherein said HMGB B box biologically active fragment consists of SEQ ID NO:23.

14. The pharmaceutical composition of claim 6, wherein said antibody is a monoclonal antibody.

15. The pharmaceutical composition of claim 6, wherein said antibody is a polyclonal antibody.

16. A method of treating a condition in a patient characterized by activation of an inflammatory cytokine cascade comprising administering to said patient a composition comprising a polypeptide comprising a high mobility group box (HMGB) A box or a fragment or variant thereof that can inhibit release of a proinflammatory cytokine from a cell treated with high mobility group box (HMGB) protein and an agent that inhibits TNF biological activity, said agent selected from the group consisting of infliximab, etanercept, adalimumab, CDP870, CDP571, Lenercept, and Thalidomide.

17. The method of claim 16, wherein said composition further comprises a pharmaceutically acceptable carrier.

18. The method of claim 16, wherein said polypeptide is a mammalian HMGB A box.

19. The method of claim 18, wherein said polypeptide is a mammalian HMGB1 A box.

20. The method of claim 19, wherein said polypeptide comprises SEQ ID NO:4.

21. The method of claim 20, wherein said polypeptide consists of SEQ ID NO:4.

22. The method of claim 16, wherein said condition is selected from the group consisting of sepsis, allograft rejection, rheumatoid arthritis, asthma, lupus, adult respiratory distress syndrome, chronic obstructive pulmonary disease, psoriasis, pancreatitis, peritonitis, burns, myocardial ischemia, organic ischemia, reperfusion ischemia, Behcet's disease, graft versus host disease, Crohn's disease, ulcerative colitis, multiple sclerosis, and cachexia.

23. A method of treating a condition in a patient characterized by activation of an inflammatory cytokine cascade comprising administering to said patient a composition comprising an antibody that binds an HMGB polypeptide or a biologically active fragment thereof and an agent that inhibits TNF biological activity, said agent selected from the group consisting of infliximab, etanercept, adalimumab, CDP870, CDP571, Lenercept, and Thalidomide.

24. The method of claim 23, wherein said composition further comprises a pharmaceutically acceptable carrier.

25. The method of claim 23, wherein said (HMGB) polypeptide is a mammalian HMGB polypeptide.

26. The method of claim 25, wherein said HMGB polypeptide is an HMGB1 polypeptide.

27. The method of claim 26, wherein said HMGB1 polypeptide comprises SEQ ID NO:1.

28. The method of claim 27, wherein said HMGB1 polypeptide consists of SEQ ID NO:1.

29. The method of claim 23, wherein said biologically active HMGB fragment is an HMGB B box or a biologically active fragment thereof.

30. The method of claim 29, wherein said HMGB1 B box consists of SEQ ID NO:5.

31. The method of claim 30, wherein said HMGB1 B box biologically active fragment consists of SEQ ID NO:23.

32. The method of claim 23, wherein said antibody is a monoclonal antibody.

33. The method of claim 23, wherein said antibody is a polyclonal antibody.

34. The method of claim 23, wherein said condition is selected from the group consisting of sepsis, allograft rejection, rheumatoid arthritis, asthma, lupus, adult respiratory distress syndrome, chronic obstructive pulmonary disease, psoriasis, pancreatitis, peritonitis, burns, myocardial ischemia, organic ischemia, reperfusion ischemia, Behcet's disease, graft versus host disease, Crohn's disease, ulcerative colitis, multiple sclerosis, and cachexia.
